SEQUENCE LISTING (3 sequences)

SEQ ID NO: 1 - DNA, 13 bp, Artificial Sequence
  Synthetic oligonucleotide
tggttatgactca

SEQ ID NO: 2 - DNA, 15 bp, Artificial Sequence
  Synthetic oligonucleotide
tttggttatgactca

SEQ ID NO: 3 - DNA, 16 bp, Artificial sequence
  Synthetic oligonucleotide
gcatgttctcacatta

(19) United States
(12) Patent Application Publication
(10) Pub. No.: US 2024/0287520 A1
(43) Pub. Date: Aug. 29, 2024

(54) METHOD FOR SYNTHESIS OF LINKAGE MODIFIED OLIGOMERIC COMPOUNDS

(71) Applicant: Ionis Pharmaceuticals, Inc., Carlsbad, CA (US)

(72) Inventor: Andrew A. Rodriguez, San Diego, CA (US)

(73) Assignee: Ionis Pharmaceuticals, Inc., Carlsbad, CA (US)

(21) Appl. No.: 18/571,631

(22) PCT Filed: Jun. 29, 2022
(86) PCT No.: PCT/US2022/035539

§ 371 Date: Dec. 18, 2023

Related U.S. Application Data
(60) Provisional application No. 63/217,137, filed on Jun. 30, 2021.

(51) Int. Cl. C12N 15/113; C07H 1/00; C07H 21/04; C12N 15/11

(52) U.S. Cl. CPC C12N 15/113; C07H 1/00; C07H 21/04; C12N 15/111; C12N 2310/11; C12N 2310/314; C12N 2310/351

(57) ABSTRACT

The present disclosure provides methods of synthesizing modified oligonucletodies and oligomeric compounds (including oligomeric compounds that are antisense agents or portions thereof) comprising a modified oligonucleotide having at least one modified internucleoside linking group. In certain embodiments, the present disclosure provides stabilized formulations of certain sulfonyl azides for use in the synthesis of olignonucleotides comprising one or more sulfonyl phosphoramidate linkages. Some embodiments provide stabilized compositions of high energy reagents that may be used in the synthesis of modified oligonucleotides, allowing for safe process scale preparation thereof.

### SEQUENCE LISTING

The present application is being filed along with a Sequence Listing in electronic format. The Sequence Listing is provided as a file entitled DVCM0048WOSEQ_ST25.txt created Jun. 29, 2022 which is 1 kb in size. The information in the electronic format of the sequence listing is incorporated herein by reference in its entirety.

### FIELD

The present disclosure provides stabilized reagent compositions for the synthesis of oligomeric compounds (including oligomeric compounds that are antisense agents or portions thereof) comprising a modified oligonucleotide having at least one modified internucleoside linking group.

### BACKGROUND

The principle behind antisense technology is that an antisense compound hybridizes to a target nucleic acid and modulates the amount, activity, and/or function of the target nucleic acid. For example, in certain instances, antisense compounds result in altered transcription or translation of a target. Such modulation of expression can be achieved by, for example, target RNA degradation or occupancy-based inhibition. An example of modulation of RNA target function by degradation is RNase H-based degradation of the target RNA upon hybridization with a DNA-like antisense compound.

Another example of modulation of gene expression by target degradation is RNA interference (RNAi). RNAi refers to antisense-mediated gene silencing through a mechanism that utilizes the RNA-induced silencing complex (RISC). An additional example of modulation of RNA target function is by an occupancy-based mechanism such as is employed naturally by microRNA. MicroRNAs are small non-coding RNAs that regulate the expression of protein-coding RNAs. The binding of an antisense compound to a microRNA prevents that microRNA from binding to its messenger RNA targets, and thus interferes with the function of the microRNA. MicroRNA mimics can enhance native microRNA function. Certain antisense compounds alter splicing of pre-mRNA. Another example of modulation of gene expression is the use of antisense compounds in a CRISPR system. Regardless of the specific mechanism, sequence-specificity makes antisense compounds attractive as tools for target validation and gene functionalization, as well as therapeutics to selectively modulate the expression of genes involved in the pathogenesis of disease.

Antisense technology is an effective means for modulating the expression of one or more specific gene products and can therefore prove to be uniquely useful in a number of therapeutic, diagnostic, and research applications. Chemically modified nucleosides may be incorporated into antisense compounds to enhance one or more properties, such as nuclease resistance, tolerability, pharmacokinetics, or affinity for a target nucleic acid. Conjugate groups may be attached to an antisense compound to enhance one or more properties, such as pharmacokinetics, pharmacodynamics, and uptake into cells and/or tissues of interest.

Oligomeric compounds comprising an oligonucleotide are chemically synthesized in a multi-step process. Substituted sulfonyl azides are useful reagents for synthesis of linkage modified oligonucleotides, but they can be high energy materials and dangerous to work with especially at the manufacturing scale. Development of new stabilized reagent compositions and reaction conditions to ameliorate potentially dangerous chemistries on scale remains an important challenge.

### SUMMARY

The present disclosure provides methods of synthesizing oligomeric compounds (including oligomeric compounds that are antisense agents or portions thereof) comprising modified oligonucleotides consisting of linked nucleosides linked through internucleoside linking groups, wherein at least one of the internucleoside linking groups has Formula I:

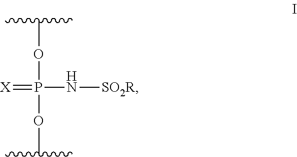

wherein X and R are as defined herein. The methods may comprise additing stabilizing materials to compositions of high energy reagents.

### DETAILED DESCRIPTION

It is to be understood that both the foregoing general description and the following detailed description are exemplary and explanatory only and are not restrictive of the embodiments, as claimed. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting.

The section headings used herein are for organizational purposes only and are not to be construed as limiting the subject matter described. All documents, or portions of documents, cited in this application, including, but not limited to, patents, patent applications, articles, books, treatises, and GenBank and NCBI reference sequence records are hereby expressly incorporated by reference for the portions of the document discussed herein, as well as in their entirety.

It is understood that the sequence set forth in each SEQ ID NO contained herein is independent of any modification to a sugar moiety, an internucleoside linkage, or a nucleobase. As such, compounds defined by a SEQ ID NO may comprise, independently, one or more modifications to a sugar moiety, an internucleoside linkage, or a nucleobase. Although the sequence listing accompanying this filing identifies each sequence as either “RNA” or “DNA” as required, in reality, those sequences may be modified with any combination of chemical modifications. One of skill in the art will readily appreciate that such designation as “RNA” or “DNA” to describe modified oligonucleotides is, in certain instances, arbitrary. For example, an oligonucleotide comprising a nucleoside comprising a 2′-OH(H) sugar moiety and a thymine base could be described as a DNA having a modified sugar (2′-OH in place of one 2′-H of DNA) or as an RNA having a modified base (thymine (methylated uracil) in place of an uracil of RNA). Accordingly, nucleic acid sequences provided herein, including, but not limited to those in the sequence listing, are intended to encompass nucleic acids containing any combination of natural or modified RNA and/or DNA, including, but not limited to such nucleic acids having modified nucleobases. By way of further example and without limitation, a modified oligonucleotide having the nucleobase sequence “ATCGATCG” encompasses any modified oligonucleotides having such nucleobase sequence, whether modified or unmodified, including, but not limited to, such compounds comprising RNA bases, such as those having sequence “AUCGAUCG” and those having some DNA bases and some RNA bases such as “AUCGATCG” and modified oligonucleotides having other modified nucleobases, such as “AT^{m}CGAUCG,” wherein ^{m}C indicates a cytosine base comprising a methyl group at the 5-position.

As used herein, “2′-substituted” in reference to a furanosyl sugar moiety or nucleoside comprising a furanosyl sugar moiety means the furanosyl sugar moiety or nucleoside comprising the furanosyl sugar moiety comprises a substituent other than H or OH at the 2′-position and is a non-bicyclic furanosyl sugar moiety. 2′-substituted furanosyl sugar moieties do not comprise additional substituents at other positions of the furanosyl sugar moiety other than a nucleobase and/or internucleoside linkage(s) when in the context of an oligonucleotide.

As used herein, “4′-substituted” in reference to a furanosyl sugar moiety or nucleoside comprising a furanosyl sugar moiety means the furanosyl sugar moiety or nucleoside comprising the furanosyl sugar moiety comprises a substituent other than H at the 4′-position and is a non-bicyclic furanosyl sugar moiety. 4′-substituted furanosyl sugar moieties do not comprise additional substituents at other positions of the furanosyl sugar moiety other than a nucleobase and/or internucleoside linkage(s) when in the context of an oligonucleotide.

As used herein, “5′-substituted” in reference to a furanosyl sugar moiety or nucleoside comprising a furanosyl sugar moiety means the furanosyl sugar moiety or nucleoside comprising the furanosyl sugar moiety comprises a substituent other than H at the 5′-position and is a non-bicyclic furanosyl sugar moiety. 5′-substituted furanosyl sugar moieties do not comprise additional substituents at other positions of the furanosyl sugar moiety other than a nucleobase and/or internucleoside linkage(s) when in the context of an oligonucleotide.

As used herein, “administration” or “administering” refers to routes of introducing a compound or composition provided herein to a subject to perform its intended function. Examples of routes of administration that can be used include, but are not limited to, administration by inhalation, subcutaneous injection, intrathecal injection, and oral administration.

As used herein, “antisense activity” means any detectable and/or measurable change attributable to the hybridization of an antisense oligonucleotide to its target nucleic acid. In certain embodiments, antisense activity is a decrease in the amount or expression of a target nucleic acid or protein encoded by such target nucleic acid compared to target nucleic acid levels or target protein levels in the absence of the antisense oligonucleotide.

As used herein, “antisense agent” means an antisense oligonucleotide or an oligonucleotide duplex comprising an antisense oligonucleotide.

As used herein, “antisense compound” means an antisense oligonucleotide or an oligonucleotide duplex comprising an antisense oligonucleotide.

As used herein, “antisense oligonucleotide” means an oligonucleotide that is complementary to a target nucleic acid and is capable of achieving at least one antisense activity. Antisense oligonucleotides include but are not limited to RNAi antisense modified oligonucleotides and RNase H antisense modified oligonucleotides. In certain embodiments, an antisense oligonucleotide is paired with a sense oligonucleotide to form an oligonucleotide duplex. In certain embodiments, an antisense oligonucleotide is unpaired and is a single-stranded antisense oligonucleotide. In certain embodiments, an antisense oligonucleotide comprises a conjugate group.

As used herein, “artificial mRNA compound” is a modified oligonucleotide, or portion thereof, having a nucleobase sequence comprising one or more codons.

As used herein, “bicyclic nucleoside” or “BNA” means a nucleoside comprising abicyclic sugar moiety. As used herein, “bicyclic sugar” or “bicyclic sugar moiety” means a modified sugar moiety comprising two rings, wherein the second ring is formed via a bridge connecting two of the atoms in the first ring thereby forming a bicyclic structure. In certain embodiments, the first ring of the bicyclic sugar moiety is a furanosyl moiety, and the bicyclic sugar moiety is a modified bicyclic furanosyl sugar moiety. In certain embodiments, the bicyclic sugar moiety does not comprise a furanosyl moiety.

As used herein, “capping reagent” means a reagent effective to protect hydroxyl groups during oligonucleotide synthesis, e.g., synthesis on a solid support. In certain embodiments, the capping reagent may be acetic anhydride. A capping reagent may be delivered in a composition including a base and a solvent. For example, a capping reagent composition may include acetic anhydride and acetonitrile, or pyridine, N-methylimidazole (NMI), and acetonitrile.

As used herein, “cEt” or “constrained ethyl” or “cEt sugar moiety” means a bicyclic sugar moiety, wherein the first ring of the bicyclic sugar moiety is a ribosyl sugar moiety, the second ring of the bicyclic sugar is formed via a bridge connecting the 4′-carbon and the 2′-carbon, the bridge has the formula 4′-CH(CH₃)—O-2′, and the methyl group of the bridge is in the S configuration. A cEt bicyclic sugar moiety is in the β-D configuration.

As used herein, “complementary” in reference to an oligonucleotide means that at least 70% of the nucleobases of such oligonucleotide or one or more regions thereof and the nucleobases of another nucleic acid or one or more regions thereof are capable of hydrogen bonding with one another when the nucleobase sequence of the oligonucleotide and the other nucleic acid are aligned in opposing directions. Complementary nucleobases are nucleobase pairs that are capable of forming hydrogen bonds with one another. Complementary nucleobase pairs include adenine (A) and thymine (T), adenine (A) and uracil (U), cytosine (C) and guanine (G), 5-methyl cytosine (^{m}C) and guanine (G). Complementary oligonucleotides and/or nucleic acids need not have nucleobase complementarity at each nucleoside. Rather, some mismatches are tolerated. As used herein, “fully complementary” or “100% complementary” in reference to oligonucleotides means that such oligonucleotides are complementary to another oligonucleotide or nucleic acid at each nucleoside of the oligonucleotide.

As used herein, “conjugate group” means a group of atoms consisting of a conjugate moiety and a conjugate linker.

As used herein, “conjugate moiety” means a group of atoms that modifies one or more properties of a molecule compared to the identical molecule lacking the conjugate moiety, including but not limited to pharmacodynamics, pharmacokinetics, stability, binding, absorption, tissue distribution, cellular distribution, cellular uptake, charge and clearance.

As used herein, “conjugate linker” means a group of atoms comprising at least one bond.

As used herein, “CRISPR compound” means a modified oligonucleotide that comprises a DNA recognition portion and a tracrRNA recognition portion. As used herein, “DNA recognition portion” is nucleobase sequence that is complementary to a DNA target. As used herein, “tracrRNA recognition portion” is a nucleobase sequence that is bound to or is capable of binding to tracrRNA. The tracrRNA recognition portion of crRNA may bind to tracrRNA via hybridization or covalent attachment.

As used herein, “cytotoxic” or “cytotoxicity” in the context of an effect of an oligomeric compound or a parent oligomeric compound on cultured cells means an at least 2-fold increase in caspase activation following administration of 10 μM or less of the oligomeric compound or parent oligomeric compound to the cultured cells relative to cells cultured under the same conditions but that are not administered the oligomeric compound or parent oligomeric compound. In certain embodiments, cytotoxicity is measured using a standard in vitro cytotoxicity assay.

As used herein, “deoxy region” means a region of 5-12 contiguous nucleotides, wherein at least 70% of the nucleosides are stereo-standard DNA nucleosides. In certain embodiments, each nucleoside is selected from a stereo-standard DNA nucleoside (a nucleoside comprising a β-D-2′-deoxyribosyl sugar moiety), a stereo-non-standard nucleoside of Formula I-VII, a bicyclic nucleoside, and a substituted stereo-standard nucleoside. In certain embodiments, a deoxy region supports RNase H activity. In certain embodiments, a deoxy region is the gap of a gapmer.

As used herein, “double-stranded antisense compound” means an antisense compound comprising two oligomeric compounds that are complementary to each other and form a duplex, and wherein one of the two said oligomeric compounds comprises an antisense oligonucleotide.

As used herein, “expression” includes all the functions by which a gene's coded information is converted into structures present and operating in a cell. Such structures include, but are not limited to, the products of transcription and translation. As used herein, “modulation of expression” means any change in amount or activity of a product of transcription or translation of a gene. Such a change may be an increase or a reduction of any amount relative to the expression level prior to the modulation.

As used herein, “gapmer” means an oligonucleotide having a central region comprising a plurality of nucleosides that support RNase H cleavage positioned between a 5′-region and a 3′-region. Herein, the nucleosides of the 5′-region and 3′-region each comprise a 2′-substituted furanosyl sugar moiety or a bicyclic sugar moiety, and the 3′- and 5′-most nucleosides of the central region each comprise a sugar moiety independently selected from a 2′-deoxyfuranosyl sugar moiety or a sugar surrogate. The positions of the central region refer to the order of the nucleosides of the central region and are counted starting from the 5′-end of the central region. Thus, the 5′-most nucleoside of the central region is at position 1 of the central region. The “central region” may be referred to as a “gap”, and the “5′-region” and “3′-region” may be referred to as “wings”. Gaps of gapmers are deoxy regions.

As used herein, “hepatotoxic” in the context of a mouse means a plasma ALT level that is above 300 units per liter. Hepatotoxicity of an oligomeric compound or parent oligomeric compound that is administered to a mouse is determined by measuring the plasma ALT level of the mouse 24 hours to 2 weeks following at least one dose of 1-150 mg/kg of the compound.

As used herein, “hepatotoxic” in the context of a human means a plasma ALT level that is above 150 units per liter. Hepatotoxicity of an oligomeric compound or parent oligomeric compound that is administered to a human is determined by measuring the plasma ALT level of the human 24 hours to 2 weeks following at least one dose of 10-300 mg of the compound.

As used herein, “hybridization” means the pairing or annealing of complementary oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding, between complementary nucleobases.

As used herein, “inhibiting the expression or activity” refers to a reduction or blockade of the expression or activity relative to the expression or activity in an untreated or control sample and does not necessarily indicate a total elimination of expression or activity.

As used herein, “internucleoside linkage” or “internucleoside linking group” means a group or bond that forms a covalent linkage between adjacent nucleosides in an oligonucleotide. As used herein “modified internucleoside linkage” means any internucleoside linkage other than a naturally occurring, phosphodiester internucleoside linkage. “Phosphorothioate linkage” means a modified internucleoside linkage in which one of the non-bridging oxygen atoms of a phosphodiester is replaced with a sulfur atom. Modified internucleoside linkages may or may not contain a phosphorus atom. A “neutral internucleoside linkage” is a modified internucleoside linkage that does not have a negatively charged phosphate in a buffered aqueous solution at pH=7.0. A modified internucleoside linkage may optionally comprise a conjugate group.

As used herein, “linked nucleosides” are nucleosides that are connected in a continuous sequence (i.e. no additional nucleosides are present between those that are linked).

As used herein, “maximum tolerated dose” means the highest dose of a compound that does not cause unacceptable side effects. In certain embodiments, the maximum tolerated dose is the highest dose of a modified oligonucleotide that does not cause an ALT elevation of three times the upper limit of normal as measured by a standard assay.

As used herein, “modulating” refers to changing or adjusting a feature in a cell, tissue, organ or organism.

As used herein, “MOE” means O-methoxyethyl. “2′-MOE” or “2′-O-methoxyethyl” means a 2′-OCH₂CH₂OCH₃ group at the 2′-position of a furanosyl ring. In certain embodiments, the 2′-OCH₂CH₂OCH₃ group is in place of the 2′-OH group of a ribosyl ring or in place of a 2′-H in a 2′-deoxyribosyl ring. A “2′-MOE sugar moiety” is a sugar moiety with a 2′-OCH₂CH₂OCH₃ group in place of the 2′—OH group of a furanosyl sugar moiety. Unless otherwise indicated, a 2′-MOE sugar moiety is in the β-D ribosyl configuration.

As used herein, a “2′-OMe sugar moiety” is a sugar moiety with a 2′-CH₃ group in place of the 2′—OH group of a furanosyl sugar moiety. Unless otherwise indicated, a 2′-OMe sugar moiety is in the β-D ribosyl configuration and is a “stereo-standard 2′OMe sugar moiety”.

As used herein, a “2′-F sugar moiety” is a sugar moiety with a 2′—F group in place of the 2′—OH group of a furanosyl sugar moiety. Unless otherwise indicated, a 2′-F sugar moiety is in the β-D ribosyl configuration and is a “stereo-standard 2′-F sugar moiety”.

As used herein, “motif” means the pattern of unmodified and/or modified sugar moieties, nucleobases, and/or internucleoside linkages, in an oligonucleotide.

As used herein, “naturally occurring” means found in nature.

As used herein, “nucleobase” means an unmodified nucleobase or a modified nucleobase. As used herein an “unmodified nucleobase” is adenine (A), thymine (T), cytosine (C), uracil (U), or guanine (G). As used herein, a modified nucleobase is a group of atoms capable of pairing with at least one unmodified nucleobase. A universal base is a nucleobase that can pair with any one of the five unmodified nucleobases. 5-methylcytosine (^{m}C) is one example of a modified nucleobase.

As used herein, “nucleobase sequence” means the order of contiguous nucleobases in a nucleic acid or oligonucleotide independent of any sugar moiety or internucleoside linkage modification.

As used herein, “nucleoside” means a moiety comprising a nucleobase and a sugar moiety. The nucleobase and sugar moiety are each, independently, unmodified or modified. As used herein, “modified nucleoside” means a nucleoside comprising a modified nucleobase and/or a modified sugar moiety. A modified nucleoside may comprise a conjugate group.

As used herein, “oligomeric compound” means a compound consisting of (1) an oligonucleotide (a single-stranded oligomeric compound) or two oligonucleotides hybridized to one another (a double-stranded oligomeric compound); and (2) optionally one or more additional features, such as a conjugate group or terminal group which may be attached to the oligonucleotide of a single-stranded oligomeric compound or to one or both oligonucleotides of a double-stranded oligomeric compound.

As used herein, “oligonucleotide” means a strand of linked nucleosides connected via internucleoside linkages, wherein each nucleoside and internucleoside linkage may be modified or unmodified. Unless otherwise indicated, oligonucleotides consist of 12-80 linked nucleosides, and optionally a conjugate group or terminal group. As used herein, “modified oligonucleotide” means an oligonucleotide, wherein at least one nucleoside (a modified nucleoside) or internucleoside linkage (a modified internucleoside linkage) is modified. As used herein, “unmodified oligonucleotide” means an oligonucleotide that does not comprise any nucleoside modifications or internucleoside modifications.

Oligonucleotides are oligomeric compounds and oligonucleotides may be incorporated into oligomeric compounds having additional features. An oligonucleotide or modified oligonucleotide may comprise a linker group that links it to a solid support. The linker may be as described in Ravikumar et al., Org. Process Res. Dev. 2008, 12, 3, 399-410.

As used herein, “oligonucleotide intermediate” means a compound or portion thereof that arises during synthesis of an oligonucleotide and that will ultimately form a portion of such oligonucleotide. Oligonucleotide intermediates include, but are not limited to linked nucleosides, internucleoside linkages, conjugate groups, and modifications described herein and precursors thereof. In certain embodiments, an oligonucleotide intermediate is a hydroxy group attached to a solid support. In certain embodiments, an oligonucleotide intermediate is a number of linked nucleosides attached to a solid support.

As used herein “pharmaceutical composition” means a mixture of substances suitable for administering to a subject. For example, a pharmaceutical composition may comprise an antisense compound and an aqueous solution.

As used herein, “RNAi agent” means an antisense agent that acts, at least in part, through RISC or Ago2 to modulate a target nucleic acid and/or protein encoded by a target nucleic acid. RNAi agents include, but are not limited to double-stranded siRNA, single-stranded RNA (ssRNA), and microRNA, including microRNA mimics. RNAi agents may comprise conjugate groups and/or terminal groups. In certain embodiments, an RNAi agent modulates the amount, activity, and/or splicing of a target nucleic acid. The term RNAi agent excludes antisense agents that act through RNase H.

As used herein, “RNAi oligonucleotide” means an RNAi antisense modified oligonucleotide or a RNAi sense modified oligonucleotide.

As used herein, “RNAi antisense modified oligonucleotide” means an oligonucleotide comprising a region that is complementary to a target sequence, and which includes at least one chemical modification suitable for RNAi.

As used herein, “RNAi antisense oligomeric compound” means a single-stranded oligomeric compound comprising a region that is complementary to a target sequence, and which includes at least one chemical modification suitable for RNAi.

As used herein, “RNAi sense modified oligonucleotide” means an oligonucleotide comprising a region that is complementary to a region of an RNAi antisense modified oligonucleotide, and which is capable of forming a duplex with such RNAi antisense modified oligonucleotide.

As used herein, “RNAi sense oligomeric compound” means a single-stranded oligomeric compound comprising a region that is complementary to a region of an RNAi antisense modified oligonucleotide and/or an RNAi antisense oligomeric compound, and which is capable of forming a duplex with such RNAi antisense modified oligonucleotide and/or RNAi antisense oligomeric compound.

A duplex formed by an RNAi antisense modified oligonucleotide and/or an RNAi antisense oligomeric compound with a RNAi sense modified oligonucleotide and/or an RNAi sense oligomeric compound is referred to as a double-stranded RNAi compound (dsRNAi) or a short interfering RNA (siRNA).

As used herein, “RNase H agent” means an antisense agent that acts, at least in part, through RNase H to modulate a target nucleic acid and/or protein encoded by a target nucleic acid. In certain embodiments, RNase H agents are single-stranded. In certain embodiments, RNase H agents are double-stranded. RNase H compounds may comprise conjugate groups and/or terminal groups. In certain embodiments, an RNase H agent modulates the amount or activity of a target nucleic acid. The term RNase H agent excludes antisense agents that act principally through RISC/Ago2.

As used herein, “RNase H antisense modified oligonucleotide” means an oligonucleotide comprising a region that is complementary to a target sequence, and which includes at least one chemical modification suitable for RNase H-mediated nucleic acid reduction.

As used herein, “RNAi compound” means an antisense compound that acts, at least in part, through RISC or Ago2 to modulate a target nucleic acid and/or protein encoded by a target nucleic acid. RNAi compounds include, but are not limited to double-stranded siRNA, single-stranded RNA (ssRNA), and microRNA, including microRNA mimics. In certain embodiments, an RNAi compound modulates the amount, activity, and/or splicing of a target nucleic acid. The term RNAi compound excludes antisense oligonucleotides that act through RNase H.

As used herein, the term “single-stranded” in reference to an antisense compound means such a compound consisting of one oligomeric compound that is not paired with a second oligomeric compound to form a duplex. “Self-complementary” in reference to an oligonucleotide means an oligonucleotide that at least partially hybridizes to itself. A compound consisting of one oligomeric compound, wherein the oligonucleotide of the oligomeric compound is self-complementary, is a single-stranded compound. A single-stranded antisense or oligomeric compound may be capable of binding to a complementary oligomeric compound to form a duplex, in which case the compound would no longer be single-stranded.

As used herein, “stabilized phosphate group” refers to a 5′-chemical moiety that results in stabilization of a 5′-phosphate moiety of the 5′-terminal nucleoside of an oligonucleotide, relative to the stability of an unmodified 5′-phosphate of an unmodified nucleoside under biologic conditions. Such stabilization of a 5′-phophate group includes but is not limited to resistance to removal by phosphatases. Stabilized phosphate groups include, but are not limited to, 5′-vinyl phosphonates and 5′-cyclopropyl phosphonate.

As used herein, “stabilizing agent” refers to a substance that when present in a solution, including but not limited to a reaction mixture, reduces the risk of explosion.

As used herein, a “standard oxidizing agent” refers to oxidizing agents well understood in the art of oligonucleotide synthesis to oxidize phosphorous internucleoside linkages, including but not limited to basic solvents, mixtures of a basic solvent such as 3-picoline, pyridine, 2,6-lutidine with Iodine and water, mixtures of Iodine, NMI, a basic solvent, and water. Further examples and description of oxidation methods are described in WO2020236618A1, the disclosure of which is incorporated in its entirety herein.

As used herein, a “standard sulfurizing agent” refers to reagents well understood in the art of oligonucleotide synthesis to sulfurize phosphorous internucleoside linkages, including but not limited to phenacetyldilsulfide or xanthane hydride. Further examples and description of oxidation methods are described in WO2020236618A1, the disclosure of which is incorporated in its entirety herein.

As used herein, “stereo-standard nucleoside” means a nucleoside comprising a non-bicyclic furanosyl sugar moiety having the configuration of naturally occurring DNA and RNA as shown below. A “stereo-standard DNA nucleoside” is a nucleoside comprising a β-D-2′-deoxyribosyl sugar moiety. A “stereo-standard RNA nucleoside” is a nucleoside comprising a β-D-ribosyl sugar moiety. A “substituted stereo-standard nucleoside” is a stereo-standard nucleoside other than a stereo-standard DNA or stereo-standard RNA nucleoside. In certain embodiments, R₁ is a 2′-substiuent and R₂-R₅ are each H. In certain embodiments, the 2′-substituent is selected from OMe, F, OCH₂CH₂OCH₃, O-alkyl, SMe, or NMA. In certain embodiments, R₁-R₄ are H and R₅ is a 5′-substituent selected from methyl, allyl, or ethyl. In certain embodiments, the heterocyclic base moiety Bx is selected from uracil, thymine, cytosine, 5-methyl cytosine, adenine or guanine. In certain embodiments, the heterocyclic base moiety Bx is other than uracil, thymine, cytosine, 5-methyl cytosine, adenine or guanine.

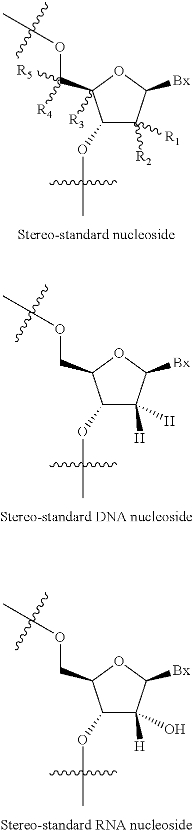

As used herein, “stereo-non-standard nucleoside” means a nucleoside comprising a non-bicyclic furanosyl sugar moiety having a configuration other than that of a stereo-standard sugar moiety. In certain embodiments, a “stereo-non-standard nucleoside” is a 2′-β-L-deoxyribosyl sugar moiety, 2′-α-D-deoxyribosyl sugar moiety, 2′-α-L-deoxyribosyl sugar moiety, a 2′-β-D-deoxyxylosyl sugar moiety, a 2′-β-L-deoxyxylosyl sugar moiety, a 2′-α-D-deoxyxylosyl sugar moiety, a 2′-α-L-deoxyxylosyl sugar moiety, a 2′-fluoro-β-D-arabinosyl sugar moiety, a 2′-fluoro-β-D-xylosyl sugar moiety, a 2′-fluoro-α-D-ribosyl sugar moiety, a 2′-fluoro-α-D-arabinosyl sugar moiety, a 2′-fluoro-α-D-xylosyl sugar moiety, a 2′-fluoro-α-L-ribosyl sugar moiety, a 2′-fluoro-β-L-xylosyl sugar moiety, a 2′-fluoro-α-L-arabinosyl sugar moiety, a 2′-fluoro-α-L-xylosyl sugar moiety, a 2′-fluoro-β-L-ribosyl sugar moiety, a 2′-fluoro-β-L-arabinosyl sugar moiety, a 2′-fluoro-β-D-lyxosyl sugar moiety, a 2′-fluoro-α-D-lyxosyl sugar moiety, a 2′-fluoro-α-L-lyxosyl sugar moiety, a 2′-fluoro-β-L-lyxosyl sugar moiety, a 2′-O-methyl-β-D-arabinosyl sugar moiety, a 2′-O-methyl-β-D-xylosyl sugar moiety, a 2′-O-methyl-α-D-ribosyl sugar moiety, a 2′-O-methyl-α-D-arabinosyl sugar moiety, a 2′-O-methyl-α-D-xylosyl sugar moiety, a 2′-O-methyl-α-L-ribosyl sugar moiety, a 2′-O-methyl-β-L-xylosyl sugar moiety, a 2′-O-methyl-α-L-arabinosyl sugar moiety, a 2′-O-methyl-α-L-xylosyl sugar moiety, a 2′-O-methyl-β-L-ribosyl sugar moiety, a 2′-O-methyl-β-L-arabinosyl sugar moiety, a 2′-O-methyl-β-D-lyxosyl sugar moiety, a 2′-O-methyl-α-D-lyxosyl sugar moiety, a 2′-O-methyl-α-L-lyxosyl sugar moiety, or a 2′-O-methyl-β-L-lyxosyl sugar moiety.

As used herein, “stereo-standard sugar moiety” means the sugar moiety of a stereo-standard nucleoside.

As used herein, “stereo-non-standard sugar moiety” means the sugar moiety of a stereo-non-standard nucleoside.

As used herein, “substituted stereo-non-standard nucleoside” means a stereo-non-standard nucleoside comprising a substituent other than the substituent corresponding to natural RNA or DNA. In certain embodiments, a substituted stereo-non-standard nucleoside is a 2′-fluoro-β-D-arabinosyl sugar moiety, a 2′-fluoro-β-D-xylosyl sugar moiety, a 2′-fluoro-α-D-ribosyl sugar moiety, a 2′-fluoro-α-D-arabinosyl sugar moiety, a 2′-fluoro-α-D-xylosyl sugar moiety, a 2′-fluoro-α-L-ribosyl sugar moiety, a 2′-fluoro-β-L-xylosyl sugar moiety, a 2′-fluoro-α-L-arabinosyl sugar moiety, a 2′-fluoro-α-L-xylosyl sugar moiety, a 2′-fluoro-β-L-ribosyl sugar moiety, a 2′-fluoro-β-L-arabinosyl sugar moiety, a 2′-fluoro-β-D-lyxosyl sugar moiety, a 2′-fluoro-α-D-lyxosyl sugar moiety, a 2′-fluoro-α-L-lyxosyl sugar moiety, a 2′-fluoro-β-L-lyxosyl sugar moiety, a 2′-O-methyl-β-D-arabinosyl sugar moiety, a 2′-O-methyl-β-D-xylosyl sugar moiety, a 2′-O-methyl-α-D-ribosyl sugar moiety, a 2′-O-methyl-α-D-arabinosyl sugar moiety, a 2′-O-methyl-α-D-xylosyl sugar moiety, a 2′-O-methyl-α-L-ribosyl sugar moiety, a 2′-O-methyl-β-L-xylosyl sugar moiety, a 2′-O-methyl-α-L-arabinosyl sugar moiety, a 2′-O-methyl-α-L-xylosyl sugar moiety, a 2′-O-methyl-β-L-ribosyl sugar moiety, a 2′-O-methyl-β-L-arabinosyl sugar moiety, a 2′-O-methyl-β-D-lyxosyl sugar moiety, a 2′-O-methyl-α-D-lyxosyl sugar moiety, a 2′-O-methyl-α-L-lyxosyl sugar moiety, or a 2′-O-methyl-β-L-lyxosyl sugar moiety.

As used herein, “sulfonyl oxidizing agent” means an agent that can effect transformation of a phosphite triester to a phosphoramidate. In certain embodiments, the sulfonyl oxidizing agent has a structure N₃—SO₂—R

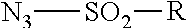

wherein R is as defined as for Formula I. In certain embodiments, R is methyl and the sulfonyl oxidizing agent is methanesulfonyl azide (“MsN₃”).

As used herein, “sugar moiety” means an unmodified sugar moiety or a modified sugar moiety. As used herein, “unmodified sugar moiety” means a β-D-ribosyl moiety, as found in naturally occurring RNA, or a β-D-2′-deoxyribosyl sugar moiety as found in naturally occurring DNA. As used herein, “modified sugar moiety” or “modified sugar” means a sugar surrogate or a furanosyl sugar moiety other than a β-D-ribosyl or a β-D-2′-deoxyribosyl. Modified furanosyl sugar moieties may be modified or substituted at a certain position(s) of the sugar moiety, or unsubstituted, and they may or may not be stereo-non-standard sugar moieties. Modified furanosyl sugar moieties include bicyclic sugars and non-bicyclic sugars. As used herein, “sugar surrogate” means a modified sugar moiety that does not comprise a furanosyl or tetrahydrofuranyl ring (is not a “furanosyl sugar moiety”) and that can link a nucleobase to another group, such as an internucleoside linkage, conjugate group, or terminal group in an oligonucleotide. Modified nucleosides comprising sugar surrogates can be incorporated into one or more positions within an oligonucleotide and such oligonucleotides are capable of hybridizing to complementary oligomeric compounds or nucleic acids.

As used herein, “target nucleic acid,” “target RNA,” “target RNA transcript” and “nucleic acid target” means a nucleic acid that an oligomeric compound, such as an antisense compound, is designed to affect. In certain embodiments, an oligomeric compound comprises an oligonucleotide having a nucleobase sequence that is complementary to more than one RNA, only one of which is the target RNA of the oligomeric compound. In certain embodiments, the target RNA is an RNA present in the species to which an oligomeric compound is administered.

As used herein, “therapeutic index” means a comparison of the amount of a compound that causes a therapeutic effect to the amount that causes toxicity. Compounds having a high therapeutic index have strong efficacy and low toxicity. In certain embodiments, increasing the therapeutic index of a compound increases the amount of the compound that can be safely administered.

### Certain Embodiments

The present disclosure provides the following non-limiting embodiments:
Embodiment 1: A method comprising contacting a first oligonucleotide intermediate having a phosphite triester internucleoside linkage with at least one stabilizing agent and with an oxidizing solution comprising a sulfonyl oxidizing agent to form a second oligonucleotide intermediate having an internucleoside linking group of Formula XIV:

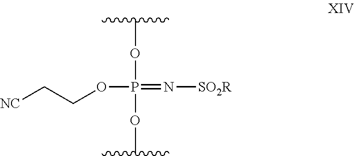

wherein:
R is selected from aryl, a substituted aryl, a heterocycle, a substituted heterocycle, an aromatic heterocycle, a substituted aromatic heterocycle, a diazole, a substituted diazole, a C₁-C₆ alkoxy, C₁-C₂₀ alkyl, C₁-C₆ alkenyl, C₁-C₆ alkynyl, substituted C₁-C₂₀ alkyl, substituted C₁-C₆ alkenyl substituted C₁-C₆ alkynyl, and a conjugate group.
Embodiment 2: The method of embodiment 1, wherein the sulfonyl oxidizing agent is methanesulfonyl azide (MsN₃).
Embodiment 3: The method of any of embodiments 1 to 2, wherein oxidizing solution comprises the at least one stabilizing agent.
Embodiment 4: The method of any of embodiments 1 to 2, wherein the oxidizing solution does not comprise the at least one stabilizing agent.
Embodiment 5: The method of any of embodiments 1 to 4, wherein the oxidizing solution comprises a solvent selected from acetonitrile, toluene, dichloromethane, pyridine, N-methyl-2-pyrrolidone, and combinations thereof.
Embodiment 6: The method of any of embodiments 1 to 5, wherein the at least one stabilizing agent is selected from naphthalene, sulfolane, and triphenylphosphate.
Embodiment 7: The method of any of embodiments 1 to 5, wherein the at least one stabilizing agent is triphenylphosphate (TPP).
Embodiment 8: The method of any of embodiments 1 to 7, wherein the at least one stabilizing agent is TPP.

### Embodiment 9

Embodiment 10: The method of any of embodiments 1 to 9, wherein at least one stabilizing agent is a non-crosslinked polymer.
Embodiment 11: The method of embodiment 10, wherein the non-crosslinked polymer is polystyrene.
Embodiment 12: The method of any of embodiments 1 to 11, wherein a residue obtained by evaporating the solvent from the oxidizing solution and the at least one stabilizing agent has combustion energy less than 500 J·g⁻¹.
Embodiment 13: The method of any of embodiments 1 to 11, wherein residue obtained by evaporating the solvent from the oxidizing solution and the at least one stabilizing agent has combustion energy less than 300 J·g⁻¹.
Embodiment 14: A method of synthesizing a modified oligonucleotide comprising at least one internucleoside linkage of Formula I:

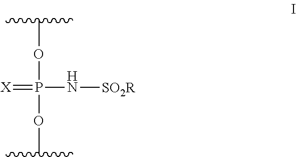

wherein independently for each internucleoside linkage of Formula I:
X is selected from O or S, and
R is selected from aryl, a substituted aryl, a heterocycle, a substituted heterocycle, an aromatic heterocycle, a substituted aromatic heterocycle, a diazole, a substituted diazole, a C₁-C₆ alkoxy, C₁-C₂₀ alkyl, C₁-C₆ alkenyl, C₁-C₆ alkynyl, substituted C₁-C₂₀ alkyl, substituted C₁-C₆ alkenyl substituted C₁-C₆ alkynyl, and a conjugate group;
wherein the method comprises steps of:
a) providing a solid support having a blocked hydroxyl group attached thereto;
b) adding a deblocking agent to the reaction to deblock the blocked hydroxyl group to provide a free hydroxyl group;
c) adding a nucleoside to the reaction to couple at the free hydroxyl group, wherein the nucleoside comprises a phosphoramidite group and a blocked hydroxyl group to provide phosphite triester linked nucleosides;
d) adding to the reaction:
   1. a standard oxidizing agent to produce a phosphate triester internucleoside linkage;
   2. a standard sulferizing agent to produce a thiophosphate triester internucleoside linkage; or
   3. a sulfonyl oxidizing agent and at least one stabilizing agent selected from TPP to produce a sulfonyl phosphoramidate internucleoside linkage;
e) optionally treating the sufonyl phosphoramidate, phosphate triester, or thiophosphate triester linkages with a mixture of capping reagents to cap any unreacted free hydroxyl groups;
f) iteratively repeating steps b) through e) a predetermined number of times to provide the modified oligonucleotide, provided that at least one iteration includes step (d) 3;
g) treating the modified oligonucleotide with triethylamine in acetonitrile; and thereby synthesizing the modified oligonucleotide comprising at least one internucleoside linkage of Formula I.
Embodiment 15: The method of embodiment 14, wherein sulfonyl oxidizing agent is in an oxidizing solution comprising methanesulfonyl azide and the at least one stabilizing agent.
Embodiment 16:
Embodiment 17: The method of any of embodiments 15 to 16, wherein the at least one stabilizing agent is TPP.
Embodiment 18: The method of any of embodiment s 14 to 17, wherein a residue obtained by evaporation of the solvent from the solution comprising the sulfonyl oxidizing agent and the at least one stabilizing agent has combustion energy less than 500 J·g⁻¹.
Embodiment 19: The method of any of embodiments 14 to 17, wherein a residue obtained by evaporation of the solvent from the solution comprising the sulfonyl oxidizing agent and the at least one stabilizing agent has combustion energy less than 300 J·g⁻¹.
Embodiment 20: The method of any of embodiments 14 to 19, wherein X is O and R is methyl.
Embodiment 21: The method of any of embodiments 1 to 20, wherein the modified oligonucleotide comprises 12 to 25 linked nucleosides.
Embodiment 22: The method of any of embodiments 14 to 21, comprising treating the modified oligonucleotide with ammonium hydroxide to remove protecting groups and cleave the modified oligonucleotide from the solid support.

### Certain Compounds

In certain embodiments, compounds described herein are oligomeric compounds (including oligomeric compounds that are antisense agents or portions thereof) comprising or consisting of oligonucleotides consisting of linked nucleosides and having at least one internucleoside linking group of Formula I:

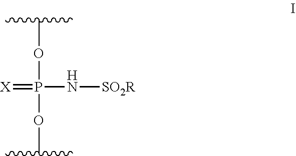

wherein X is selected from O or S, and
R is selected from aryl, a substituted aryl, a heterocycle, a substituted heterocycle, an aromatic heterocycle, a substituted aromatic heterocycle, a diazole, a substituted diazole, a C₁-C₆ alkoxy, C₁-C₂₀ alkyl, C₁-C₆ alkenyl, C₁-C₆ alkynyl, substituted C₁-C₂₀ alkyl, substituted C₁-C₆ alkenyl substituted C₁-C₆ alkynyl, and a conjugate group. In certain embodiments, X is O and R is methyl, and the internucleoside linking group of Formula I is an internucleoside linking group of Formula II below.

In certain embodiments, compounds described herein are oligomeric compounds (including oligomeric compounds that are antisense agents or portions thereof) comprising or consisting of oligonucleotides consisting of linked nucleosides and having at least one internucleoside linking group of Formula II:

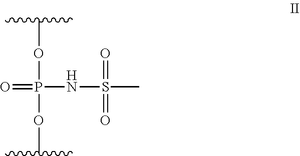 [SMILES: *OP(=O)(NS(C)(=O)=O)O*]

(a mesyl phosphoramidate internucleoside linkage).

In certain embodiments, compounds described herein are oligomeric compounds (including oligomeric compounds that are antisense agents or portions thereof) comprising or consisting of oligonucleotides consisting of linked nucleosides and having at least one internucleoside linking group of Formula III:

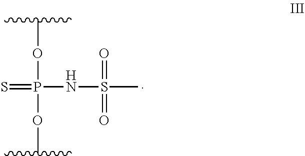 [SMILES: *OP(=S)(NS(C)(=O)=O)O*]

In certain embodiments, compounds described herein are oligomeric compounds (including oligomeric compounds that are antisense agents or portions thereof) comprising or consisting of oligonucleotides consisting of linked nucleosides and having at least one internucleoside linking group of Formula IV:

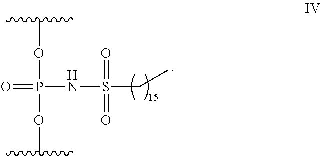

Modified oligonucleotides comprise at least one modification relative to an unmodified oligonucleotide (i.e., comprise at least one modified nucleoside (comprising a modified sugar moiety, a stereo-non-standard nucleoside, and/or a modified nucleobase) and/or at least one modified internucleoside linkage). In certain embodiments, the modified internucleoside linkage is a modified internucleoside linking group having any of Formula I-IV. In certain embodiments, compounds described herein are oligomeric compounds (including oligomeric compounds that are antisense agents or portions thereof) having at least one modified internucleoside linking group having any of Formula I-IV.

### Certain Process for the Synthesis of Oligonucleotides

The present disclosure provides synthetic methods for preparing oligonucleotides comprising at least one modified internucleoside linkage of the Formula I:

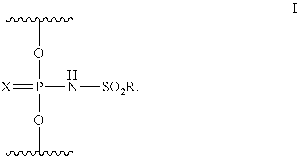

The present disclosure also provides synthetic methods for preparing oligomeric compounds comprising such oligonucleotides, where such oligomeric compounds comprise a conjugate moiety attached to the oligonucleotide through a cleavable linker. In certain embodiments, the cleavable linker is a phosphate diester bond. In certain embodiments, oligonucleotides having both at least one internucleoside linkage of Formula I and at least one phosphorothioate diester linkage and/or at least one phosphate diester linkage have one or more desired properties. In certain embodiments, oligonucleotides having at least one internucleoside linkage of the Formula I are gapmers. In certain embodiments, oligonucleotides having at least one internucleoside linkage of the Formula I are used to modulate splicing of a nucleic acid target. In certain embodiments, oligonucleotides having at least one internucleoside linkage of the Formula I are RNAi compounds. Such RNAi compounds may be double-stranded or single-stranded. Such oligonucleotides may comprise any of the features, modified nucleosides, and nucleoside motifs described herein.

Accordingly, such oligonucleotides may comprise any of the modified sugar moieties described herein and/or any of the modified nucleobases. In certain embodiments, the synthetic processes described herein are used to synthesize oligomeric compounds comprising a conjugate group. In certain embodiments, the synthetic processes described herein are used to synthesize oligomeric compounds comprising a conjugate group comprising one or more N-Acetylgalactosamine residues. In certain embodiments, the oligomeric compounds synthesized using the processes described herein are gapmers. In certain embodiments, oligomeric compounds synthesized using the processes described herein are RNAi compounds. In certain embodiments, oligomeric compounds synthesized using the processes described herein are single-stranded. In certain embodiments, oligomeric compounds synthesized using the processes described herein are double-stranded. In certain embodiments, compounds synthesized using the processes described herein are formulated for administration to an animal.

### Certain Reagents for the Synthesis of Oligonucleotides Comprising Sulfonyl Phosphoramidate Internucleoside Linkages

The present disclosure provides certain sulfonyl oxidizing agents, for example, sulfonyl azides, for use in the synthesis of olignonucleotides comprising one or more sulfonyl phosphoramidate linkages. The present disclosure further provides stabilizing agents that may be introduced to sulfonyl phosphoramidate linkage formation reactions. In certain embodiments, the stabilizing agents may be introduced to the linkage formation reaction before, concomitantly with, or subsequent to introduction of the sulfonyl oxidizing agent, for example, sulfonyl azide. In certain embodiments, more than one stabilizing agent may be introduced to the linkage formation reactions. In certain embodiments, the stabilizing agents may ameliorate energetic events associated with use of sulfonyl azides. In certain embodiments, the stabilizing agents may be sterically bulky compounds. In certain embodiments, the stabilizing agents have low flammability properties. In certain embodiments, the stabilizing agents have low volatility and may be solid or semi-solid at room temperature. In certain embodiments, the sulfonyl azide and stabilizing agents may be dissolved in a solvent or solvent mixture prior to introduction to the linkage formation reaction. Solvents that may be useful according to the present disclosure include, but are not limited to acetonitrile (MeCN), dichloromethane (DCM), toluene, pyridine, N-methyl-2-pyrrolidone (NMP), and mixtures thereof. Solvents that may be useful according to the present disclosure include, but are not limited to acetonitrile (MeCN), toluene, dichloromethane (DCM), toluene, pyridine, N-methyl-2-pyrrolidone (NMP), and mixtures thereof. In certain embodiments, the solvents are acetonitrile and toluene. In certain embodiments, the stabilizing agents may be solids or liquids at room temperature. Stabilizing agents that are useful in the present disclosure include, but are not limited to naphthalene, sulfolane, and triphenylphosphate (TPP). In certain embodiments, stabilizing agents for use in the present disclosure are non-crosslinked polymers, including but not limited to polystyrene. Additional stabilizing agents contemplated herein include soluble polymers, waxes, triglycerides, and paraffin wax.

A stabilizing agent should form a homogenous mixture with the sulfonyl oxidizing agent upon evaporation of a solvent. Stabilizing agents that are prone to crystallization are not believed to be suitable. Thus, diphenyl sulfone (DPS) was found to form crystals and is not suitable.

In general, a stabilizing agent described herein reduces the exotherm created during reaction of a sulfonyl oxidizing agent, such as methanesulfonyl azide. The amount of stabilizing agent relative to the amount of sulfonyl oxidizing agent may be determined. In certain embodiments, the amount of stabilizing agent provides a composition that can be handled and utilized in synthesis safely. The stabilizing agent may be in an amount that permits synthesis of oligonucleotides on process scale, for example, oligonucleotides may be synthesized in amounts sufficient to carry out clinical trials. The stabilizing agent may be easily removable by solvent or aqueous wash. The stabilizing agent, as used in synthesis of therapeutic oligonucleotides, may be compatible with GMP protocols. In certain embodiments, the stabilizing agent provides a composition that does not have a risk of explosion upon impact, for example, when methanesulfonyl azide is the sulfonyl oxidizing agent.

Also provided herein is a stabilized composition comprising methanesulfonyl azide and a stabilizing agent. In certain embodiments, provided is a stabilized composition comprising, consisting essentially, or consisting of sulfolane and methansulfonyl azide. The stabilized composition may further comprise a solvent. In certain embodiments, provided is a stabilized composition comprising, consisting essentially, or consisting of sulfolane, methansulfonyl azide, and optionally acetonitrile. The stabilized composition may optionally be placed in contact with a solid support carrying an oligonucleotide intermediate for use in the synthesis of oligomeric compounds that contain phosphoramidate internucleoside linkages, as described herein.

In certain embodiments, processes described herein are useful for synthesizing oligomeric compounds comprising or consisting of oligonucleotides consisting of linked nucleosides. The present disclosure provides reagents for use in the synthesis of oligonucleotides having any number of modifications described herein.

Thus, provided in certain embodiments is a stabilized composition comprising methanesulfonyl azide and sulfolane. The composition may comprise 0.1 to 100 equivalents, 0.1 to 10 equivalents, 1 to 10 equivalents, 3 to 6 equivalents, 4 to 5 equivalents, 1 to 2 equivalents, 2 to 3 equivalents, 3 to 4 equivalents, 5 to 6 equivalents, 6 to 7 equivalents, 7 to 8 equivalents, 8 to 9 equivalents, or 9 to 10 equivalents of sulfolane relative to methanesulfonyl azide, or about 1, 2, 3, 4, 5, 6, 7, 8, 9, or 10 equivalents of sulfolane relative to methanesulfonyl azide. The concentration of methanesulfonyl azide in the stabilized composition may be 0.1 to 10 M, for example 0.5 to 10 M, 0.5 to 5 M, 0.1 to 5 M, 0.3 to 1.5 M, 0.4 to 0.6 M, 0.1 to 0.2 M, 0.2 to 0.3 M, 0.3 to 0.4 M, 0.4 to 0.5 M, 0.4 to 0.6 M, 0.5 to 0.6 M, 0.6 to 0.7 M, 0.7 to 0.8 M, 0.8 to 0.9 M, 0.9 to 1 M, 1 to 1.1 M, 1 to 1.2 M, 1.1 to 1.2 M, 1.1 to 1.3 M, 1.2 to 1.3 M, 1 to 1.5 M, 1.3 to 1.4 M, 1.4 to 1.5 M, or about 0.1, 0.2, 0.3, 0.4, 0.5, 0.6, 0.7, 0.8, 0.9, 1, 1.1, 1.2, 1.3, 1.4, 1.5, or 2 M. The concentration of sulfolane in the stabilized composition may be 0.1 to 20 M, for example 1 to 20 M, 1 to 10 M, 3 to 6 M, 4 to 5 M, 1 to 2 M, 2 to 3 M, 3 to 4 M, 5 to 6 M, 6 to 7 M, 7 to 8 M, 8 to 9 M, or 9 to 10 M, or about 1, 2, 3, 4, 5, 6, 7, 8, 9, or 10 M.

### I. Modifications

### A. Modified Nucleosides

Modified nucleosides comprise a stereo-non-standard nucleoside, or a modified sugar moiety, or a modified nucleobase, or any combination thereof.

### 1. Certain Modified Sugar Moieties

In certain embodiments, modified sugar moieties are stereo-non-standard sugar moieties. In certain embodiments, sugar moieties are substituted furanosyl stereo-standard sugar moieties. In certain embodiments, modified sugar moieties are bicyclic or tricyclic furanosyl sugar moieties. In certain embodiments, modified sugar moieties are sugar surrogates. Such sugar surrogates may comprise one or more substitutions corresponding to those of other types of modified sugar moieties.

a. Stereo-Non-Standard Sugar Moieties

In certain embodiments, modified sugar moieties are stereo-non-standard sugar moieties shown in Formulas V-XI below:

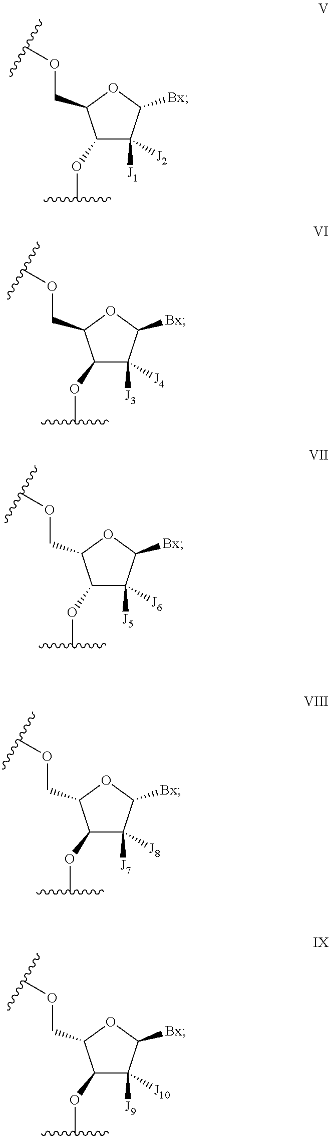

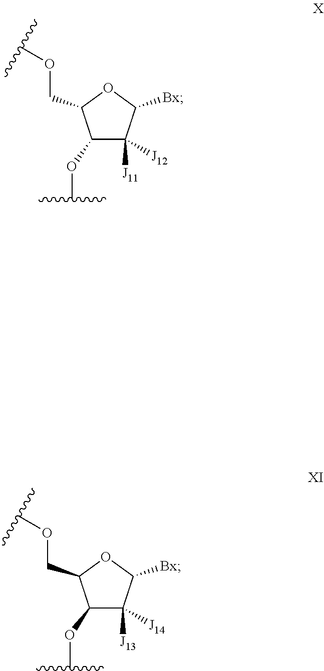

wherein
one of J₁ and J₂ is H and the other of J₁ and J₂ is selected from H, OH, F, OCH₃, OCH₂CH₂OCH₃, O—C₁-C₆ alkoxy, and SCH₃;
one of J₃ and J₄ is H and the other of J₃ and J₄ is selected from H, OH, F, OCH₃, OCH₂CH₂OCH₃, O—C₁-C₆ alkoxy, and SCH₃; and wherein
one of J₅ and J₆ is H and the other of J₅ and J₆ is selected from H, OH, F, OCH₃, OCH₂CH₂OCH₃, O—C₁-C₆ alkoxy, and SCH₃; and wherein
one of J₇ and J₈ is H and the other of J₇ and J₈ is selected from H, OH, F, OCH₃, OCH₂CH₂OCH₃, O—C₁-C₆ alkoxy, and SCH₃; and wherein
one of J₉ and J₁₀ is H and the other of J₉ and J₁₀ is selected from H, OH, F, OCH₃, OCH₂CH₂OCH₃, O—C₁-C₆ alkoxy, and SCH₃; and wherein
one of J₁₁ and J₁₂ is H and the other of J₁₁ and J₁₂ is selected from H, OH, F, OCH₃, OCH₂CH₂OCH₃, O—C₁-C₆ alkoxy, and SCH₃; and wherein
one of J₁₃ and J₁₄ is H and the other of J₁₃ and J₁₄ is selected from H, OH, F, OCH₃, OCH₂CH₂OCH₃, O—C₁-C₆ alkoxy, and SCH₃; and
Bx is a is a heterocyclic base moiety.

Certain stereo-non-standard sugar moieties have been previously described in, e.g., Seth et al., WO2020/072991 and Seth et al., WO2019/157531, both of which are incorporated by reference herein in their entirety.

b. Substituted Stereo-Standard Sugar Moieties

In certain embodiments, modified sugar moieties are substituted stereo-standard furanosyl sugar moieties comprising one or more acyclic substituent, including but not limited to substituents at the 2′, 3′, 4′, and/or 5′ positions. In certain embodiments, the furanosyl sugar moiety is a ribosyl sugar moiety. In certain embodiments one or more acyclic substituent of substituted stereo-standard sugar moieties is branched. Examples of 2′-substituent groups suitable for substituted stereo-standard sugar moieties include but are not limited to: 2′-F, 2′-OCH₃ (“2′-OMe” or “2′-O-methyl”), and 2′-O(CH₂)₂OCH₃ (“2′-MOE”). In certain embodiments, 2′-substituent groups are selected from among: halo, allyl, amino, azido, SH, CN, OCN, CF₃, OCF₃, O—C₁-C₁₀ alkoxy, O—C₁-C₁₀ substituted alkoxy, C₁-C₁₀ alkyl, C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂ON(Rₘ)(Rₙ) or OCH₂C(═O)—N(Rₘ)(Rₙ), where each Rₘ and Rₙ is, independently, H, an amino protecting group, or substituted or unsubstituted C₁-C₁₀ alkyl, and the 2′-substituent groups described in Cook et al., U.S. Pat. No. 6,531,584; Cook et al., U.S. Pat. No. 5,859,221; and Cook et al., U.S. Pat. No. 6,005,087. Certain embodiments of these 2′-substituent groups can be further substituted with one or more substituent groups independently selected from among: hydroxyl, amino, alkoxy, carboxy, benzyl, phenyl, nitro (NO₂), thiol, thioalkoxy, thioalkyl, halogen, alkyl, aryl, alkenyl and alkynyl. Examples of 3′-substituent groups include 3′-methyl (see Frier, et al., The ups and downs of nucleic acid duplex stability: structure-stability studies on chemically-modified DNA:RNA duplexes. Nucleic Acids Res., 25, 4429-4443, 1997.) Examples of 4′-substituent groups suitable for substituted stereo-standard sugar moieties include but are not limited to alkoxy (e.g., methoxy), alkyl, and those described in Manoharan et al., WO 2015/106128. Examples of 5′-substituent groups suitable for substituted stereo-standard sugar moieties include but are not limited to: 5′-methyl (R or S), 5′-allyl, 5′-ethyl, 5′-vinyl, and 5′-methoxy. In certain embodiments, non-bicyclic modified sugars comprise more than one non-bridging sugar substituent, for example, 2′-F-5′-methyl sugar moieties and the modified sugar moieties and modified nucleosides described in Migawa et al., WO 2008/101157 and Rajeev et al., US2013/0203836. 2′,4′-difluoro modified sugar moieties have been described in Martinez-Montero, et al., Rigid 2′,4′-difluororibonucleosides: synthesis, conformational analysis, and incorporation into nascent RNA by HCV polymerase. J. Org. Chem., 2014, 79:5627-5635. Modified sugar moieties comprising a 2′-modification (OMe or F) and a 4′-modification (OMe or F) have also been described in Malek-Adamian, et al., J. Org. Chem, 2018, 83: 9839-9849.

In certain embodiments, a 2′-substituted stereo-standard nucleoside comprises a sugar moiety comprising a non-bridging 2′-substituent group selected from: F, NH₂, N₃, OCF₃, OCH₃, SCH₃, O(CH₂)₃NH₂, CH₂CH═CH₂, OCH₂CH═CH₂, OCH₂CH₂OCH₃, O(CH₂)₂SCH₃, O(CH₂)₂ON(Rₘ)(Rₙ), O(CH₂)₂O(CH₂)₂N(CH₃)₂, and N-substituted acetamide (OCH₂C(═O)—N(Rₘ)(Rₙ)), where each Rₘ and Rₙ is, independently, H, an amino protecting group, or substituted or unsubstituted C₁-C₁₀ alkyl.

In certain embodiments, a 2′-substituted stereo-standard nucleoside comprises a sugar moiety comprising a non-bridging 2′-substituent group selected from: F, OCF₃, OCH₃, OCH₂CH₂OCH₃, O(CH₂)₂SCH₃, O(CH₂)₂ON(CH₃)₂, O(CH₂)₂O(CH₂)₂N(CH₃)₂, and OCH₂C(═O)—N(H)CH₃ (“NMA”).

In certain embodiments, a 2′-substituted stereo-standard nucleoside comprises a sugar moiety comprising a 2′-substituent group selected from: F, OCH₃, and OCH₂CH₂OCH₃.

In certain embodiments, the 4′ 0 of 2′-deoxyribose can be substituted with a S to generate 4′-thio DNA (see Takahashi, et al., Nucleic Acids Research 2009, 37: 1353-1362). This modification can be combined with other modifications detailed herein. In certain such embodiments, the sugar moiety is further modified at the 2′ position. In certain embodiments the sugar moiety comprises a 2′-fluoro. A thymidine with this sugar moiety has been described in Watts, et al., J. Org. Chem. 2006, 71(3): 921-925 (4′-S-fluoro5-methylarauridine or FAMU).

c. Bicyclic Nucleosides

Certain nucleosides comprise modified sugar moieties that comprise a bridging sugar substituent that forms a second ring resulting in a bicyclic sugar moiety. In certain such embodiments, the bicyclic sugar moiety comprises a 4′ to 2′ bridge between the 4′ and the 2′ furanose ring atoms. In certain such embodiments, the furanose ring is a ribose ring. Examples of sugar moieties comprising such 4′ to 2′ bridging sugar substituents include but are not limited to bicyclic sugars comprising: 4′-CH₂-2′, 4′-(CH₂)₂-2′, 4′-(CH₂)₃-2′, 4′-CH₂—O-2′ (“LNA”), 4′-CH₂—S-2′, 4′-(CH₂)₂-O-2′ (“ENA”), 4′-CH(CH₃)—O-2′ (referred to as “constrained ethyl” or “cEt” when in the S configuration), 4′-CH₂—O—CH₂-2′, 4′-CH₂—N(R)-2′, 4′-CH(CH₂OCH₃)—O-2′ (“constrained MOE” or “cMOE”) and analogs thereof (see, e.g., Seth et al., U.S. Pat. No. 7,399,845, Bhat et al., U.S. Pat. No. 7,569,686, Swayze et al., U.S. Pat. No. 7,741,457, and Swayze et al., U.S. Pat. No. 8,022,193), 4′-C(CH₃)(CH₃)—O-2′ and analogs thereof (see, e.g., Seth et al., U.S. Pat. No. 8,278,283), 4′-CH₂—N(OCH₃)-2′ and analogs thereof (see, e.g., Prakash et al., U.S. Pat. No. 8,278,425), 4′-CH₂—O—N(CH₃)-2′ (see, e.g., Allerson et al., U.S. Pat. No. 7,696,345 and Allerson et al., U.S. Pat. No. 8,124,745), 4′-CH₂—C(H)(CH₃)-2′ (see, e.g., Zhou, et al., J. Org. Chem., 2009, 74, 118-134), 4′-CH₂—C(═CH₂)-2′ and analogs thereof (see e.g., Seth et al., U.S. Pat. No. 8,278,426), 4′-C(RₐR_{b})—N(R)—O-2′, 4′—C(RₐR_{b})—O—N(R)-2′, 4′-CH₂—O—N(R)-2′, and 4′- CH₂—N(R)—O-2′, wherein each R, Rₐ, and R_{b} is, independently, H, a protecting group, or C₁-C₁₂ alkyl (see, e.g. Imanishi et al., U.S. Pat. No. 7,427,672), 4′-C(═O)—N(CH₃)₂-2′, 4′-C(═O)—N(R)₂-2′, 4′-C(═S)—N(R)₂-2′ and analogs thereof (see, e.g., Obika et al., WO2011052436A1, Yusuke, WO2017018360A1).

Additional bicyclic sugar moieties are known in the art, see, for example: Freier et al., Nucleic Acids Research, 1997, 25(22), 4429-4443, Albaek et al., J. Org. Chem., 2006, 71, 7731-7740, Singh et al., Chem. Commun., 1998, 4, 455-456; Koshkin et al., Tetrahedron, 1998, 54, 3607-3630; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 2017, 129, 8362-8379; Elayadi et al.; Christiansen, et al., J. Am. Chem. Soc. 1998, 120, 5458-5463; Wengel et al., U.S. Pat. No. 7,053,207; Imanishi et al., U.S. Pat. No. 6,268,490; Imanishi et al. U.S. Pat. No. 6,770,748; Imanishi et al., U.S. RE44,779; Wengel et al., U.S. Pat. No. 6,794,499; Wengel et al., U.S. Pat. No. 6,670,461; Wengel et al., U.S. Pat. No. 7,034,133; Wengel et al., U.S. Pat. No. 8,080,644; Wengel et al., U.S. Pat. No. 8,034,909; Wengel et al., U.S. Pat. No. 8,153,365; Wengel et al., U.S. Pat. No. 7,572,582; and Ramasamy et al., U.S. Pat. No. 6,525,191; Torsten et al., WO 2004/106356; Wengel et al., WO 1999/014226; Seth et al., WO 2007/134181; Seth et al., U.S. Pat. No. 7,547,684; Seth et al., U.S. Pat. No. 7,666,854; Seth et al., U.S. Pat. No. 8,088,746; Seth et al., U.S. Pat. No. 7,750,131; Seth et al., U.S. Pat. No. 8,030,467; Seth et al., U.S. Pat. No. 8,268,980; Seth et al., U.S. Pat. No. 8,546,556; Seth et al., U.S. Pat. No. 8,530,640; Migawa et al., U.S. Pat. No. 9,012,421; Seth et al., U.S. Pat. No. 8,501,805; and U.S. patenttent Publication Nos. Allerson et al., US2008/0039618 and Migawa et al., US2015/0191727.

In certain embodiments, bicyclic sugar moieties and nucleosides incorporating such bicyclic sugar moieties are further defined by isomeric configuration. For example, an LNA nucleoside (described herein) may be in the α-L configuration or in the β-D configuration.

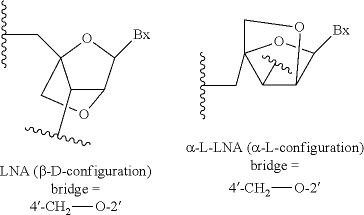

α-L-methyleneoxy (4′-CH₂—O-2′) or α-L-LNA bicyclic nucleosides have been incorporated into antisense oligonucleotides that showed antisense activity (Frieden et al., Nucleic Acids Research, 2003, 21, 6365-6372). Herein, general descriptions of bicyclic nucleosides include both isomeric configurations. When the positions of specific bicyclic nucleosides (e.g., LNA) are identified in exemplified embodiments herein, they are in the β-D configuration, unless otherwise specified.

In certain embodiments, modified sugar moieties comprise one or more non-bridging sugar substituent and one or more bridging sugar substituent (e.g., 5′-substituted and 4′-2′ bridged sugars).

The term “substituted” following a position of the furanosyl ring, such as “2′-substituted” or “2′-4′-substituted”, indicates that is the only position(s) having a substituent other than those found in unmodified sugar moieties in oligonucleotides.

d. Sugar Surrogates

In certain embodiments, modified sugar moieties are sugar surrogates. In certain such embodiments, the oxygen atom of the sugar moiety is replaced, e.g., with a sulfur, carbon or nitrogen atom. In certain such embodiments, such modified sugar moieties also comprise bridging and/or non-bridging substituents as described herein. For example, certain sugar surrogates comprise a 4′-sulfur atom and a substitution at the 2′-position (see, e.g., Bhat et al., U.S. Pat. No. 7,875,733 and Bhat et al., U.S. Pat. No. 7,939,677) and/or the 5′ position.

In certain embodiments, sugar surrogates comprise rings having other than 5 atoms. For example, in certain embodiments, a sugar surrogate comprises a six-membered tetrahydropyran (“THP”). Such tetrahydropyrans may be further modified or substituted. Nucleosides comprising such modified tetrahydropyrans include but are not limited to hexitol nucleic acid (“HNA”), altritol nucleic acid (“ANA”), mannitol nucleic acid (“INA”) (see, e.g., Leumann, CJ. Bioorg. & Med. Chem. 2002, 10, 841-854), fluoro HNA (“F-HNA”, see e.g.Swayze et al., U.S. Pat. No. 8,088,904; Swayze et al., U.S. Pat. No. 8,440,803; Swayze et al., U.S. Pat. No. 8,796,437; and Swayze et al., U.S. Pat. No. 9,005,906; F-HNA can also be referred to as a F-THP or 3′-fluoro tetrahydropyran).

In certain embodiments, sugar surrogates comprise rings having no heteroatoms. For example, nucleosides comprising bicyclo [3.1.0]-hexane have been described (see, e.g., Marquez, et al., J. Med. Chem. 1996, 39:3739-3749).

In certain embodiments, sugar surrogates comprise rings having more than 5 atoms and more than one heteroatom. For example, nucleosides comprising morpholino sugar moieties and their use in oligonucleotides have been reported (see, e.g., Braasch et al., Biochemistry, 2002, 41, 4503-4510 and Summerton et al., U.S. Pat. No. 5,698,685; Summerton et al., U.S. Pat. No. 5,166,315; Summerton et al., U.S. Pat. No. 5,185,444; and Summerton et al., U.S. Pat. No. 5,034,506). As used here, the term “morpholino” means a sugar surrogate comprising the following structure:

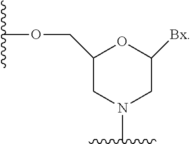

In certain embodiments, morpholinos may be modified, for example by adding or altering various substituent groups from the above morpholino structure. Such sugar surrogates are referred to herein as “modified morpholinos.” In certain embodiments, morpholino residues replace a full nucleotide, including the internucleoside linkage, and have the structures shown below, wherein Bx is a heterocyclic base moiety.

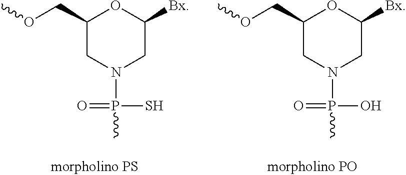

In certain embodiments, sugar surrogates comprise acyclic moieites. Examples of nucleosides and oligonucleotides comprising such acyclic sugar surrogates include but are not limited to: peptide nucleic acid (“PNA”), acyclic butyl nucleic acid (see, e.g., Kumar et al., Org. Biomol. Chem., 2013, 11, 5853-5865), glycol nucleic acid (“GNA”, see Schlegel, et al., J. Am. Chem. Soc. 2017, 139:8537-8546) and nucleosides and oligonucleotides described in Manoharan et al., WO2011/133876. In certain embodiments, acyclic sugar surrogates are selected from:

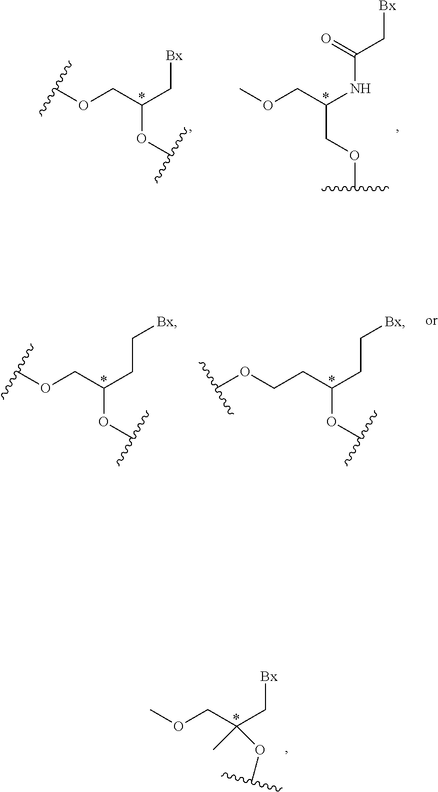

Many other bicyclic and tricyclic sugar and sugar surrogate ring systems are known in the art that can be used in modified nucleosides. Certain such ring systems are described in Hanessian, et al., J Org. Chem., 2013, 78: 9051-9063 and include bcDNA and tcDNA. Modifications to bcDNA and tcDNA, such as 6′-fluoro, have also been described (Dogovic and Leumann, J. Org. Chem., 2014, 79: 1271-1279).

e. Conjugated Nucleosides and Terminal Groups

In certain embodiments, modified sugar moieties comprise a conjugate group and/or a terminal group. Modified sugar moieties are linked to conjugate groups through a conjugate linker. In certain embodiments, modified furanosyl sugar moieties comprise conjugate groups attached at the 2′, 3′, or 5′ positions. In certain embodiments, the 3′-most sugar moiety of the nucleoside is modified with a conjugate group or a terminal group. In certain embodiments, the 5′-most sugar moiety of the nucleoside is modified with a conjugate group or a terminal group. In certain embodiments, a sugar moiety near the 3′ end of the nucleoside is modified with a conjugate group. In certain embodiments, a sugar moiety near the 5′ end of the nucleoside is modified with a conjugate group.

Examples of terminal groups include but are not limited to conjugate groups, capping groups, phosphate group, protecting groups, modified or unmodified nucleosides, and two or more nucleosides that are independently modified or unmodified.

In certain embodiments, terminal groups at the 5′-terminus comprise a stabilized phosphate group. In certain such embodiments, the phosphorus atom of the stabilized phosphate group is attached to the 5′-terminal nucleoside through a phosphorus-carbon bond. In certain embodiments, the carbon of that phosphorus-carbon bond is in turn bound to the 5′-position of the nucleoside.

In certain embodiments, the oligonucleotide comprises a 5′-stabilized phosphate group having the following formula:

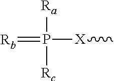

wherein:
Rₐ and R_{c} are each, independently, OH, SH, C₁-C₆ alkyl, substituted C₁-C₆ alkyl, C₁-C₆ alkoxy, substituted C₁-C₆ alkoxy, amino or substituted amino;
R_{b} is O or S;
X is substituted or unsubstituted C; and wherein X is attached to the 5′-terminal nucleoside. In certain embodiments, X is bound to an atom at the 5′-position of the 5′-terminal nucleoside. In certain such embodiments, the 5′-atom is a carbon and the bond between X and the 5′-carbon of the 5′-terminal nucleoside is a carbon-carbon single bond. In certain embodiments, it is a carbon-carbon double bond. In certain embodiments, it is a carbon-carbon triple bond. In certain embodiments, the 5′-carbon is substituted. In certain embodiments, X is substituted. In certain embodiments, X is unsubstituted.

In certain embodiments, the oligonucleotide comprises a 5′-stabilized phosphate group having the following formula:

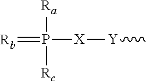

wherein:
Rₐ and R_{c} are each, independently, OH, SH, C₁-C₆ alkyl, substituted C₁-C₆ alkyl, C₁-C₆ alkoxy, substituted C₁-C₆ alkoxy, amino or substituted amino;
R_{b} is O or S;
X is substituted or unsubstituted C;
Y is selected from C, S, and N. In certain embodiments, Y is substituted or unsubstituted C. The bond between X and Y may be a single-, double-, or triple-bond.

Certain 5′-stabilized phosphate groups have been previously described; see, e.g., Prakash et al., WO2011/139699 and Prakash et al., WO2011/139702, hereby incorporated by reference herein in their entirety.

In certain embodiments, the stabilized phosphate group is 5′-vinyl phosphonate or 5′-cyclopropyl phosphonate.

In certain embodiments, a terminal group at the 5′-terminus is a 5′-mesyl phosphoramidate, having formula XII:

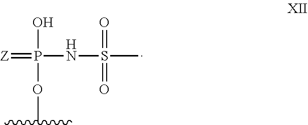

wherein Z is O or S.

In certain embodiments, a terminal group at the 5′-terminus is a 5′-mesyl phosphoramidate, having formula XIII:

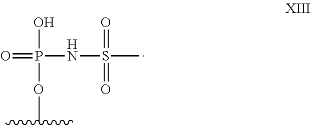 [SMILES: *OP(=O)(O)NS(C)(=O)=O]

### 2. Modified Nucleobases

In certain embodiments, modified nucleobases are selected from: 5-substituted pyrimidines, 6-azapyrimidines, alkyl or alkynyl substituted pyrimidines, alkyl substituted purines, and N-2, N-6 and 0-6 substituted purines. In certain embodiments, modified nucleobases are selected from: 2-aminopropyladenine, 5-hydroxymethyl cytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (—C═C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in Merigan et al., U.S. Pat. No. 3,687,808, those disclosed in The Concise Encyclopedia of Polymer Science And Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443. In certain embodiments, modified nucleosides comprise double-headed nucleosides having two nucleobases. Such compounds are described in detail in Sorinas et al., J. Org. Chem, 2014 79: 8020-8030.

Publications that teach the preparation of certain of the above noted modified nucleobases as well as other modified nucleobases include without limitation, Manoharan et al., US2003/0158403; Manoharan et al., US2003/0175906; Dinh et al., U.S. Pat. No. 4,845,205; Spielvogel et al., U.S. Pat. No. 5,130,302; Rogers et al., U.S. Pat. No. 5,134,066; Bischofberger et al., U.S. Pat. No. 5,175,273; Urdea et al., U.S. Pat. No. 5,367,066; Benner et al., U.S. Pat. No. 5,432,272; Matteucci et al., U.S. Pat. No. 5,434,257; Gmeiner et al., U.S. Pat. No. 5,457,187; Cook et al., U.S. Pat. No. 5,459,255; Froehler et al., U.S. Pat. No. 5,484,908; Matteucci et al., U.S. Pat. No. 5,502,177; Hawkins et al., U.S. Pat. No. 5,525,711; Haralambidis et al., U.S. Pat. No. 5,552,540; Cook et al., U.S. Pat. No. 5,587,469; Froehler et al., U.S. Pat. No. 5,594,121; Switzer et al., U.S. Pat. No. 5,596,091; Cook et al., U.S. Pat. No. 5,614,617; Froehler et al., U.S. Pat. No. 5,645,985; Cook et al., U.S. Pat. No. 5,681,941; Cook et al., U.S. Pat. No. 5,811,534; Cook et al., U.S. Pat. No. 5,750,692; Cook et al., U.S. Pat. No. 5,948,903; Cook et al., U.S. Pat. No. 5,587,470; Cook et al., U.S. Pat. No. 5,457,191; Matteucci et al., U.S. Pat. No. 5,763,588; Froehler et al., U.S. Pat. No. 5,830,653; Cook et al., U.S. Pat. No. 5,808,027; Cook et al., 6,166,199; and Matteucci et al., U.S. Pat. No. 6,005,096.

In certain embodiments, compounds comprise or consist of a modified oligonucleotide complementary to a target nucleic acid comprising one or more modified nucleobases. In certain embodiments, the modified nucleobase is 5-methylcytosine. In certain embodiments, each cytosine is a 5-methylcytosine.

### B. Modified Internucleoside Linkages

a. Internucleoside Linkages of Formula I

In certain embodiments, antisense agents, oligomeric compounds, and modified oligonucleotides described herein having one or more modified internucleoside linkages having Formula I are selected over compounds lacking such internucleoside linkages having Formula I because of one or more desirable properties. In certain embodiments, antisense agents, oligomeric compounds, and modified oligonucleotides described herein having one or more modified internucleoside linkages having Formula I have enhanced cellular uptake. In certain embodiments, antisense agents, oligomeric compounds, and modified oligonucleotides described herein having one or more modified internucleoside linkages having Formula I have enhanced affinity for target nucleic acids. In certain embodiments, antisense agents, oligomeric compounds, and modified oligonucleotides described herein having one or more modified internucleoside linkages having Formula I have increased stability in the presence of nucleases. In certain embodiments, antisense agents, oligomeric compounds, and modified oligonucleotides described herein having one or more modified internucleoside linkages having Formula I have enhanced cellular uptake, enhanced affinity for target nucleic acids, and increased stability in the presence of nucleases. In certain embodiments, antisense agents, oligomeric compounds, and modified oligonucleotides described herein having one or more modified internucleoside linkages having Formula I have enhanced bioavailability. In certain embodiments, antisense agents, oligomeric compounds, and modified oligonucleotides described herein having one or more modified internucleoside linkages having Formula I have enhanced RNase H activity. In certain embodiments, antisense agents, oligomeric compounds, and modified oligonucleotides described herein having one or more modified internucleoside linkages having Formula I have enhanced RNAi activity. In certain embodiments, antisense agents, oligomeric compounds, and modified oligonucleotides described herein having one or more modified internucleoside linkages having Formula I have enhanced CRISPR activity. In certain embodiments, antisense agents, oligomeric compounds, and modified oligonucleotides described herein having one or more modified internucleoside linkages having Formula I have reduced interactions with certain proteins. In certain embodiments, antisense agents, oligomeric compounds, and modified oligonucleotides described herein having one or more modified internucleoside linkages having Formula I have increased interactions with certain proteins. Methods of making oligonucleotides having at least one internucleoside linkage of Formula I (including but not limited to Formula II-IV) may be used to make oligomeric compounds having any of the above properties.

In certain embodiments, oligomeric compounds (including oligomeric compounds that are antisense agents or portions thereof) comprise or consist of a modified oligonucleotide complementary to a target nucleic acid comprising one or more modified internucleoside linkages having Formula I:

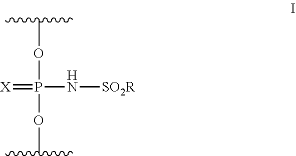

wherein independently for each internucleoside linkage of Formula I:
X is selected from O or S, and
R is selected from aryl, a substituted aryl, a heterocycle, a substituted heterocycle, an aromatic heterocycle, a substituted aromatic heterocycle, a diazole, a substituted diazole, a C₁-C₆ alkoxy, C₁-C₂₀ alkyl, C₁-C₆ alkenyl, C₁-C₆ alkynyl, substituted C₁-C₂₀ alkyl, substituted C₁-C₆ alkenyl substituted C₁-C₆ alkynyl, and a conjugate group.

### Other Internucleoside Linkages

In certain embodiments, antisense agents, oligomeric compounds, and modified oligonucleotides comprise one or more internucleoside linkages of Formula I and one or more internucleoside linkages that are not of Formula I. In certain embodiments, such internucleoside linkages are phosphorothioate linkages. In certain embodiments, each internucleoside linkage of an oligomeric compound other than the at least one internucleoside linkage of Formula I is a phosphorothioate internucleoside linkage. In certain embodiments, each internucleoside linkage of an oligomeric compound other than the at least one internucleoside linkage of Formula I is a phosphorothioate internucleoside linkage or a phosphodieser internucleoside linkage.

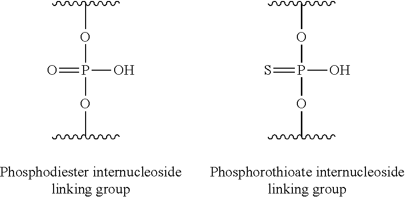 [SMILES: *OP(=O)(O)O* ; *OP(O)(=S)O*]

In certain embodiments, nucleosides of modified oligonucleotides may be linked together using any internucleoside linkage. The two main classes of internucleoside linkages are defined by the presence or absence of a phosphorus atom. Representative phosphorus-containing internucleoside linkages include unmodified phosphodiester internucleoside linkages, modified phosphotriesters such as THP phosphotriester and isopropyl phosphotriester, phosphonates such as methylphosphonate, isopropyl phosphonate, isobutyl phosphonate, and phosphonoacetate, phosphoramidates, phosphorothioate, and phosphorodithioate (“HS—P═S”). Representative non-phosphorus containing internucleoside linkages include but are not limited to methylenemethylimino (—CH₂—N(CH₃)—O—CH₂—), thiodiester, thionocarbamate (—O—C(═O)(NH)—S—); siloxane (—O—SiH₂—O—); formacetal, thioacetamido (TANA), alt-thioformacetal, glycine amide, and N,N′-dimethylhydrazine (—CH₂—N(CH₃)—N(CH₃)—). Modified internucleoside linkages, compared to naturally occurring phosphate linkages, can be used to alter, typically increase, nuclease resistance of the oligonucleotide. Methods of preparation of phosphorous-containing and non-phosphorous-containing internucleoside linkages are well known to those skilled in the art.

Neutral internucleoside linkages include, without limitation, phosphotriesters, phosphonates, MMI (3′-CH₂—N(CH₃)—O—5′), amide-3 (3′-CH₂—C(═O)—N(H)-5′), amide-4 (3′-CH₂—N(H)—C(═O)-5′), formacetal (3′-O—CH₂—O—5′), methoxypropyl, and thioformacetal (3′-S—CH₂—O—5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH2 component parts.

b. Chiral Internucleoside Linkages

Representative internucleoside linkages having a chiral center include but are not limited to alkylphosphonates and phosphorothioates. Modified oligonucleotides comprising internucleoside linkages having a chiral center can be prepared as populations of modified oligonucleotides comprising stereorandom internucleoside linkages, or as populations of modified oligonucleotides comprising phosphorothioate linkages in particular stereochemical configurations. In certain embodiments, populations of modified oligonucleotides comprise phosphorothioate internucleoside linkages wherein all of the phosphorothioate internucleoside linkages are stereorandom. Such modified oligonucleotides can be generated using synthetic methods that result in random selection of the stereochemical configuration of each phosphorothioate linkage. All phosphorothioate linkages described herein are stereorandom unless otherwise specified. Nonetheless, as is well understood by those of skill in the art, each individual phosphorothioate of each individual oligonucleotide molecule has a defined stereoconfiguration. In certain embodiments, populations of modified oligonucleotides are enriched for modified oligonucleotides comprising one or more particular phosphorothioate internucleoside linkages in a particular, independently selected stereochemical configuration. In certain embodiments, the particular configuration of the particular phosphorothioate linkage is present in at least 65% of the molecules in the population. In certain embodiments, the particular configuration of the particular phosphorothioate linkage is present in at least 70% of the molecules in the population. In certain embodiments, the particular configuration of the particular phosphorothioate linkage is present in at least 80% of the molecules in the population. In certain embodiments, the particular configuration of the particular phosphorothioate linkage is present in at least 90% of the molecules in the population. In certain embodiments, the particular configuration of the particular phosphorothioate linkage is present in at least 99% of the molecules in the population. Such chirally enriched populations of modified oligonucleotides can be generated using synthetic methods known in the art, e.g., methods described in Oka et al., JACS 125, 8307 (2003), Wan et al. Nuc. Acid. Res. 42, 13456 (2014), and WO 2017/015555. In certain embodiments, a population of modified oligonucleotides is enriched for modified oligonucleotides having at least one indicated phosphorothioate in the (Sp) configuration. In certain embodiments, a population of modified oligonucleotides is enriched for modified oligonucleotides having at least one phosphorothioate in the (Rp) configuration. In certain embodiments, modified oligonucleotides comprising (Rp) and/or (Sp) phosphorothioates comprise one or more of the following formulas, respectively, wherein “B” indicates a nucleobase:

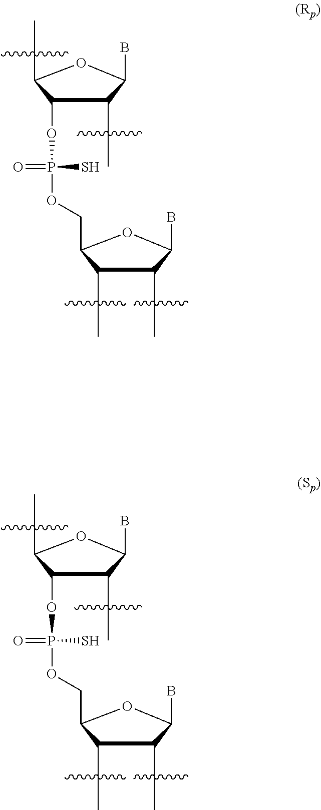

Unless otherwise indicated, chiral internucleoside linkages of modified oligonucleotides described herein can be stereorandom or in a particular stereochemical configuration.

In certain embodiments, an internucleoside linkage of Formula I may comprise a chiral center. In certain embodiments, modified oligonucleotides comprise chiral linkages of Formula II, as shown below.

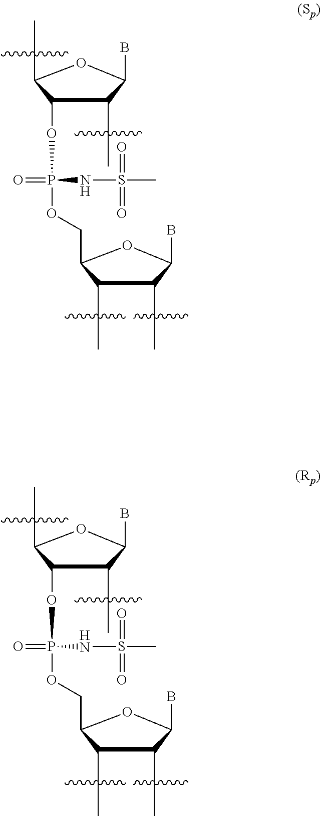

c. Alternatives to 5′ to 3′ Internucleoside Linkages

In certain embodiments, nucleic acids can be linked 2′ to 5′ rather than the standard 3′ to 5′ linkage. Such a linkage is illustrated below.

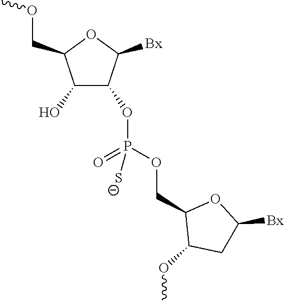

In certain embodiments, nucleosides can be linked by 2′, 3′-phosphodiester bonds. In certain such embodiments, the nucleosides are threofuranosyl nucleosides (TNA; see Bala, et al., J Org. Chem. 2017, 82:5910-5916). A TNA linkage is shown below.

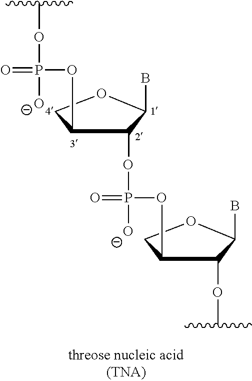

Additional modified linkages include α,β-D-CN A type linkages and related comformationally-constrained linkages, shown below, Synthesis of such molecules has been described previously (see Dupouy, et al., Angew. Chem. Int. Ed. Engl., 2014, 45: 3623-3627; Borsting, et al, Tetrahedron, 2004, 60:10955-10966; Ostergaard, et al., ACS Chem. Biol. 2014, 9: 1975-197: Dupouy, et al., Eur. J. Org Chem., 2008, 1285-1294; Martinez, et al., PLoS One, 2011, 6: e25510; Dupouy, et al., Eur. J Org. Chem., 2007, 5256-5264; Boissonnet, et al., New J. Chem., 2011, 35: 1528-1533.)

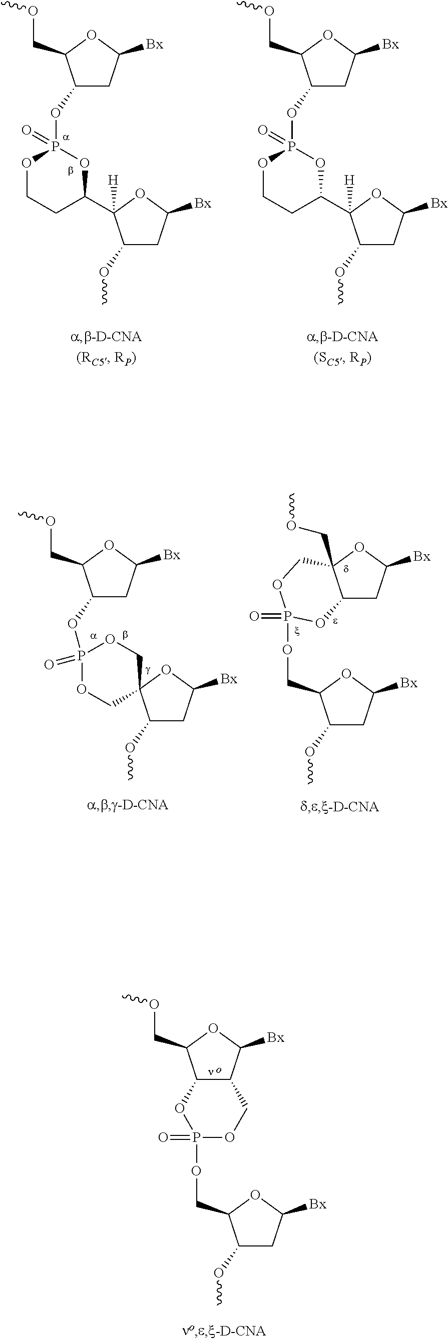

d. Linkages Having Conjugate Groups

In certain embodiments, an internucleoside linking group may comprise a conjugate group. In certain embodiments, an internucleoside linking group of Formula I comprises a conjugate group. In certain embodiments, the conjugate group of a modified oligonucleotide may be attached to the remainder of the modified oligonucleotide through a modified internucleoside having Formula I:

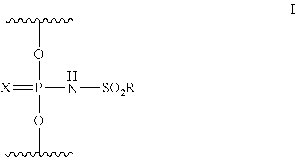

wherein R comprises a conjugate group. In certain embodiments, the conjugate group comprises a cell-targeting moiety. In certain embodiments, the conjugate group comprises a carbohydrate or carbohydrate cluster. In certain embodiments, the conjugate group comprises N-acetylgalactosamine (GalNAc). In certain embodiments, the conjugate group comprises a lipid. In certain embodiments, the conjugate group comprises C₁₀-C₂₀ alkyl. In certain embodiments, the conjugate group comprises C₁₆ alkyl.

In certain embodiments, the internucleoside linking group comprising a conjugate group has Formula IV:

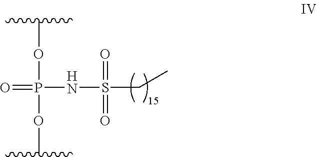

### II. Certain Motifs

In certain embodiments, antisense agents, oligomeric compounds, and modified oligonucleotides described herein comprise or consist of oligonucleotides. Modified oligonucleotides can be described by their motif, e.g. a pattern of unmodified and/or modified sugar moieties, nucleobases, and/or internucleoside linkages. In certain embodiments, modified oligonucleotides comprise one or more stereo-non-standard nucleosides. In certain embodiments, modified oligonucleotides comprise one or more stereo-standard nucleosides. In certain embodiments, modified oligonucleotides comprise one or more modified nucleoside comprising a modified sugar. In certain embodiments, modified oligonucleotides comprise one or more modified nucleosides comprising a modified nucleobase. In certain embodiments, modified oligonucleotides comprise one or more modified internucleoside linkage. In such embodiments, the modified, unmodified, and differently modified sugar moieties, nucleobases, and/or internucleoside linkages of a modified oligonucleotide define a pattern or motif. In certain embodiments, the patterns or motifs of sugar moieties, nucleobases, and internucleoside linkages are each independent of one another. Thus, a modified oligonucleotide may be described by its sugar motif, nucleobase motif and/or internucleoside linkage motif (as used herein, nucleobase motif describes the modifications to the nucleobases independent of the sequence of nucleobases).

### A. Certain Sugar Motifs

In certain embodiments, antisense agents, oligomeric compounds, and modified oligonucleotides described herein comprise or consist of oligonucleotides. In certain embodiments, oligonucleotides comprise one or more type of modified sugar and/or unmodified sugar moiety arranged along the oligonucleotide or region thereof in a defined pattern or sugar motif. In certain instances, such sugar motifs include without limitation any of the sugar modifications discussed herein.

In certain embodiments, a modified oligonucleotide comprises or consists of a gapmer. The sugar motif of a gapmer defines the regions of the gapmer: 5′-region, central region (gap), and 3′-region. The central region is linked directly to the 5′-region and to the 3′-region with no nucleosides intervening. The central region is a deoxy region. The nucleoside at the first position (position 1) from the 5′-end of the central region and the nucleoside at the last position of the central region are adjacent to the 5′-region and 3′-region, respectively, and each comprise a sugar moiety independently selected from a 2′-deoxyfuranosyl sugar moiety or a sugar surrogate. In certain embodiments, the nucleoside at position 1 of the central region and the nucleoside at the last position of the central region are DNA nucleosides, selected from stereo-standard DNA nucleosides or stereo-non-standard DNA nucleosides having any of formulas I-VII, wherein each J is H. In certain embodiments, the nucleoside at the first and last positions of the central region adjacent to the 5′ and 3′ regions are stereo-standard DNA nucleosides. Unlike the nucleosides at the first and last positions of the central region, the nucleosides at the other positions within the central region may comprise a 2′-substituted furanosyl sugar moiety or a substituted stereo-non-standard sugar moiety or a bicyclic sugar moiety. In certain embodiments, each nucleoside within the central region supports RNase H cleavage. In certain embodiments, a plurality of nucleosides within the central region support RNase H cleavage.

Herein, the lengths (number of nucleosides) of the three regions of a gapmer may be provided using the notation [#of nucleosides in the 5′-region]-[#of nucleosides in the central region]-[#of nucleosides in the 3′-region]. Thus, a 3-10-3 gapmer consists of 3 linked nucleosides in each of the 3′ and 5′ regions and 10 linked nucleosides in the central region. Where such nomenclature is followed by a specific modification, that modification is the modification of each sugar moiety of each 5′ and 3′-region and the central region nucleosides comprise stereo-standard DNA sugar moieties. Thus, a 5-10-5 MOE gapmer consists of 5 linked nucleosides each comprising 2′-MOE-stereo-standard sugar moieties in the 5′-region, 10 linked nucleosides each comprising a stereo-standard DNA sugar moiety in the central region, and 5 linked nucleosides each comprising 2′-MOE-stereo-standard sugar moieties in the 3′-region. A 5-10-5 MOE gapmer having a substituted stereo-non-standard nucleoside at position 2 of the gap has a gap of 10 nucleosides wherein the 2^{nd} a nucleoside of the gap is a substituted stereo-non-standard nucleoside rather than the stereo-standard DNA nucleoside. Such oligonucleotide may also be described as a 5-1-1-8-5 MOE/substituted stereo-non-standard/MOE gapmer. A 3-10-3 cEt gapmer consists of 3 linked nucleosides each comprising a cEt in the 5′-region, 10 linked nucleosides each comprising a stereo-standard DNA sugar moiety in the central region, and 3 linked nucleosides each comprising a cEt in the 3′-region. A 3-10-3 cEt gapmer having a substituted stereo-non-standard nucleoside at position 2 of the gap has a gap of 10 nucleoside wherein the 2^{nd} nucleoside of the gap is a substituted stereo-non-standard nucleoside rather than the stereo-standard DNA nucleoside. Such oligonucleotide may also be described as a 3-1-1-8-3 cEt/substituted stereo-non-standard/cEt gapmer.

The sugar motif of a 3-10-3 cEt gapmer may also be denoted by the notation kkk-d(10)-kkk, wherein each “k” represents a cEt and each “d” represents a 2′-β-D-deoxyribosyl sugar moiety. This sugar motif is independent of the nucleobase sequence, the internucleoside linkage motif, and any nucleobase modifications. A 5-10-5 MOE gapmer may be denoted by the notation eeeee-d(10)-eeeee or e(5)-d(10)-e(5), wherein each “e” represents a 2′-MOE-β-D-ribofuranosyl sugar moiety, and each “d” represents a 2′-β-D-deoxyribosyl sugar moiety.

In certain embodiments, each nucleoside of a modified oligonucleotide, or portion thereof, comprises a 2′-substituted sugar moiety, a bicyclic sugar moiety, a sugar surrogate, or a 2′-deoxyribosyl sugar moiety. In certain embodiments, the 2′-substituted sugar moiety is selected from a 2′-MOE sugar moiety, a 2′-NMA sugar moiety, a 2′-OMe sugar moiety, and a 2′-F sugar moiety. In certain embodiments, the bicyclic sugar moiety is selected from a cEt sugar moiety and an LNA sugar moiety. In certain embodiments, the sugar surrogate is selected from morpholino, modified morpholino, PNA, THP, and F-HNA.

In certain embodiments, modified oligonucleotides comprise at least 12, at least 13, at least 14, at least 15, at least 16, at least 17, at least 18, at least 19, or at least 20 nucleosides comprising a modified sugar moiety. In certain embodiments, the modified sugar moiety is selected independently from a 2′-substituted sugar moiety, a bicyclic sugar moiety, or a sugar surrogate. In certain embodiments, the 2′-substituted sugar moiety is selected from a 2′-MOE sugar moiety, a 2′-NMA sugar moiety, a 2′-OMe sugar moiety, and a 2′-F sugar moiety. In certain embodiments, the bicyclic sugar moiety is selected from a cEt sugar moiety and an LNA sugar moiety. In certain embodiments, the sugar surrogate is selected from morpholino, modified morpholino, THP, and F-HNA.

In certain embodiments, each nucleoside of a modified oligonucleotide comprises a modified sugar moiety (“fully modified oligonucleotide”). In certain embodiments, each nucleoside of a fully modified oligonucleotide comprises a 2′-substituted sugar moiety, abicyclic sugar moiety, or a sugar surrogate. In certain embodiments, the 2′-substituted sugar moiety is selected from a 2′-MOE sugar moiety, a 2′-NMA sugar moiety, a 2′-OMe sugar moiety, and a 2′-F sugar moiety. In certain embodiments, the bicyclic sugar moiety is selected from a cEt sugar moiety and an LNA sugar moiety. In certain embodiments, the sugar surrogate is selected from morpholino, modified morpholino, THP, and F-HNA. In certain embodiments, each nucleoside of a fully modified oligonucleotide comprises the same modified sugar moiety (“uniformly modified sugar motif”). In certain embodiments, the uniformly modified sugar motif is 7 to 20 nucleosides in length. In certain embodiments, each nucleoside of the uniformly modified sugar motif comprises a 2′-substituted sugar moiety, a bicyclic sugar moiety, or a sugar surrogate. In certain embodiments, the 2′-substituted sugar moiety is selected from a 2′-MOE sugar moiety, a 2′-NMA sugar moiety, a 2′-OMe sugar moiety, and a 2′-F sugar moiety. In certain embodiments, the bicyclic sugar moiety is selected from a cEt sugar moiety and an LNA sugar moiety. In certain embodiments, the sugar surrogate is selected from morpholino, modified morpholino, THP, and F-HNA. In certain embodiments, modified oligonucleotides having at least one fully modified sugar motif may also comprise at least 1, at least 2, at least 3, or at least 4 2′-deoxyribonucleosides.

### B. Certain Nucleobase Motifs

In certain embodiments antisense agents, oligomeric compounds, and modified oligonucleotides described herein comprise or consist of oligonucleotides. In certain embodiments, oligonucleotides comprise modified and/or unmodified nucleobases arranged along the oligonucleotide or region thereof in a defined pattern or motif. In certain embodiments, each nucleobase is modified. In certain embodiments, none of the nucleobases are modified. In certain embodiments, each purine or each pyrimidine is modified. In certain embodiments, each adenine is modified. In certain embodiments, each guanine is modified. In certain embodiments, each thymine is modified. In certain embodiments, each uracil is modified. In certain embodiments, each cytosine is modified. In certain embodiments, some or all of the cytosine nucleobases in a modified oligonucleotide are 5-methylcytosines.

In certain embodiments, modified oligonucleotides comprise a block of modified nucleobases. In certain such embodiments, the block is at the 3′-end of the oligonucleotide. In certain embodiments the block is within 3 nucleosides of the 3′-end of the oligonucleotide. In certain embodiments, the block is at the 5′-end of the oligonucleotide. In certain embodiments the block is within 3 nucleosides of the 5′-end of the oligonucleotide.

In certain embodiments, one nucleoside comprising a modified nucleobase is in the central region of a modified oligonucleotide. In certain such embodiments, the sugar moiety of said nucleoside is a 2′-β-D-deoxyribosyl moiety. In certain such embodiments, the modified nucleobase is selected from: 5-methyl cytosine, 2-thiopyrimidine, 2-thiothymine, 6-methyladenine, inosine, pseudouracil, or 5-propynepyrimidine.

### C. Certain Internucleoside Linkage Motifs

In certain embodiments, antisense agents, oligomeric compounds, and modified oligonucleotides described herein comprise or consist of oligonucleotides. In certain embodiments, oligonucleotides comprise modified and/or unmodified internucleoside linkages arranged along the oligonucleotide or region thereof in a defined pattern or motif.

In certain embodiments, the one or two 5′-most internucleoside linkages are internucleoside linkages of Formula I. In certain embodiments, the one or two 3′-most internucleoside linkages are internucleoside linkages of Formula I. In certain embodiments, each internucleoside linkage is selected from an internucleoside linkage of Formula I, a phosphorothioate internucleoside linkage, and a phosphodiester internucleoside linkage. In certain embodiments, each internucleoside linkage is selected from an internucleoside linkage of Formula I and a phosphodiester internucleoside linkage.

In certain embodiments, each phosphorothioate internucleoside linkage is independently selected from a stereorandom phosphorothioate, a (Sp) phosphorothioate, and a (Rp) phosphorothioate. In certain embodiments, the internucleoside linkages within the central region of a modified oligonucleotide are all modified. In certain such embodiments, all of the phosphorothioate linkages are stereorandom. In certain embodiments, all of the phosphorothioate linkages in the 5′-region and 3′-region are (Sp) phosphorothioates, and the central region comprises at least one Sp, Sp, Rp motif. In certain embodiments, populations of modified oligonucleotides are enriched for modified oligonucleotides comprising such internucleoside linkage motifs.

In certain embodiments, a double-stranded antisense compound is a double-stranded RNAi compound comprising an RNAi antisense modified oligonucleotide and an RNAi sense modified oligonucleotide, wherein one or both of the RNAi antisense modified oligonucleotide and/or RNAi sense oligomeric compound have one or more modified internucleoside linking groups having Formula I. In certain embodiments, the RNAi antisense modified oligonucleotide comprises at least two, at least three, at least four, at least five, or at least six modified internucleoside linking groups having Formula I. In certain embodiments, the RNAi sense modified oligonucleotide comprises at least two, at least three, at least four, at least five, or at least six modified internucleoside linking groups having Formula I.

In certain embodiments, the RNAi antisense modified oligonucleotide comprises exactly one modified internucleoside linking group having Formula I. In certain embodiments, the RNAi antisense modified oligonucleotide comprises exactly two modified internucleoside linking groups having Formula I. In certain embodiments, the RNAi antisense modified oligonucleotide comprises exactly three modified internucleoside linking groups having Formula I.

In certain embodiments, the RNAi antisense modified oligonucleotide comprises exactly four modified internucleoside linking groups having Formula I.

In certain embodiments, the RNAi sense modified oligonucleotide comprises exactly one modified internucleoside linking group having Formula I. In certain embodiments, the RNAi sense modified oligonucleotide comprises exactly two modified internucleoside linking groups having Formula I. In certain embodiments, the RNAi sense modified oligonucleotide comprises exactly three modified internucleoside linking groups having Formula I. In certain embodiments, the RNAi sense modified oligonucleotide comprises exactly four modified internucleoside linking groups having Formula I. In certain embodiments, the RNAi sense modified oligonucleotide comprises exactly five modified internucleoside linking groups having Formula I.

In certain embodiments, at least one of the five 3′-most internucleoside linking groups of the RNAi antisense modified oligonucleotide is a modified internucleoside linking group having Formula I. In certain embodiments, at least two of the five 3′-most internucleoside linking groups of the RNAi antisense modified oligonucleotide are modified internucleoside linking groups having Formula I.

### D. Certain Modified Oligonucleotides

In certain embodiments, antisense agents, oligomeric compounds, and modified oligonucleotides described herein comprise or consist of modified oligonucleotides. In certain embodiments, the above modifications (sugar, nucleobase, internucleoside linkage) are incorporated into a modified oligonucleotide. In certain embodiments, modified oligonucleotides are characterized by their modifications, motifs, and overall lengths. In certain embodiments, such parameters are each independent of one another. Thus, unless otherwise indicated, each internucleoside linkage of a modified oligonucleotide may be modified or unmodified and may or may not follow the modification pattern of the sugar moieties. Likewise, such modified oligonucleotides may comprise one or more modified nucleobase independent of the pattern of the sugar modifications. Furthermore, in certain instances, a modified oligonucleotide is described by an overall length or range and by lengths or length ranges of two or more regions (e.g., a region of nucleosides having specified sugar modifications), in such circumstances it may be possible to select numbers for each range that result in an oligonucleotide having an overall length falling outside the specified range. In such circumstances, both elements must be satisfied. For example, in certain embodiments, a modified oligonucleotide consists of 15-20 linked nucleosides and has a sugar motif consisting of three regions or segments, A, B, and C, wherein region or segment A consists of 2-6 linked nucleosides having a specified sugar moiety, region or segment B consists of 6-10 linked nucleosides having a specified sugar moiety, and region or segment C consists of 2-6 linked nucleosides having a specified sugar moiety. Such embodiments do not include modified oligonucleotides where A and C each consist of 6 linked nucleosides and B consists of 10 linked nucleosides (even though those numbers of nucleosides are permitted within the requirements for A, B, and C) because the overall length of such oligonucleotide is 22, which exceeds the upper limit of 20 for the overall length of the modified oligonucleotide. Unless otherwise indicated, all modifications are independent of nucleobase sequence except that the modified nucleobase 5-methylcytosine is necessarily a “C” in an oligonucleotide sequence. In certain embodiments, when a DNA nucleoside or DNA-like nucleoside that comprises a T in a DNA sequence is replaced with a RNA-like nucleoside, the nucleobase T is replaced with the nucleobase U. Each of these compounds has an identical target RNA.

In certain embodiments, oligonucleotides consist of X to Y linked nucleosides, where X represents the fewest number of nucleosides in the range and Y represents the largest number nucleosides in the range. In certain such embodiments, X and Y are each independently selected from 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50; provided that X≤Y. For example, in certain embodiments, oligonucleotides consist of 12 to 13, 12 to 14, 12 to 15, 12 to 16, 12 to 17, 12 to 18, 12 to 19, 12 to 20, 12 to 21, 12 to 22, 12 to 23, 12 to 24, 12 to 25, 12 to 26, 12 to 27, 12 to 28, 12 to 29, 12 to 30, 13 to 14, 13 to 15, 13 to 16, 13 to 17, 13 to 18, 13 to 19, 13 to 20, 13 to 21, 13 to 22, 13 to 23, 13 to 24, 13 to 25, 13 to 26, 13 to 27, 13 to 28, 13 to 29, 13 to 30, 14 to 15, 14 to 16, 14 to 17, 14 to 18, 14 to 19, 14 to 20, 14 to 21, 14 to 22, 14 to 23, 14 to 24, 14 to 25, 14 to 26, 14 to 27, 14 to 28, 14 to 29, 14 to 30, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 16 to 17, 16 to 18, 16 to 19, 16 to 20, 16 to 21, 16 to 22, 16 to 23, 16 to 24, 16 to 25, 16 to 26, 16 to 27, 16 to 28, 16 to 29, 16 to 30, 17 to 18, 17 to 19, 17 to 20, 17 to 21, 17 to 22, 17 to 23, 17 to 24, 17 to 25, 17 to 26, 17 to 27, 17 to 28, 17 to 29, 17 to 30, 18 to 19, 18 to 20, 18 to 21, 18 to 22, 18 to 23, 18 to 24, 18 to 25, 18 to 26, 18 to 27, 18 to 28, 18 to 29, 18 to 30, 19 to 20, 19 to 21, 19 to 22, 19 to 23, 19 to 24, 19 to 25, 19 to 26, 19 to 29, 19 to 28, 19 to 29, 19 to 30, 20 to 21, 20 to 22, 20 to 23, 20 to 24, 20 to 25, 20 to 26, 20 to 27, 20 to 28, 20 to 29, 20 to 30, 21 to 22, 21 to 23, 21 to 24, 21 to 25, 21 to 26, 21 to 27, 21 to 28, 21 to 29, 21 to 30, 22 to 23, 22 to 24, 22 to 25, 22 to 26, 22 to 27, 22 to 28, 22 to 29, 22 to 30, 23 to 24, 23 to 25, 23 to 26, 23 to 27, 23 to 28, 23 to 29, 23 to 30, 24 to 25, 24 to 26, 24 to 27, 24 to 28, 24 to 29, 24 to 30, 25 to 26, 25 to 27, 25 to 28, 25 to 29, 25 to 30, 26 to 27, 26 to 28, 26 to 29, 26 to 30, 27 to 28, 27 to 29, 27 to 30, 28 to 29, 28 to 30, or 29 to 30 linked nucleosides.

In certain embodiments oligonucleotides have a nucleobase sequence that is complementary to a second oligonucleotide or an identified reference nucleic acid, such as a target nucleic acid. In certain embodiments, a region of an oligonucleotide has a nucleobase sequence that is complementary to a second oligonucleotide or an identified reference nucleic acid, such as a target nucleic acid. In certain embodiments, the nucleobase sequence of a region or entire length of an oligonucleotide is at least 70%, at least 80%, at least 90%, at least 95%, or 100% complementary to the second oligonucleotide or nucleic acid, such as a target nucleic acid.

### III. Certain Conjugated Compounds

In certain embodiments, antisense agents, oligomeric compounds, and modified oligonucleotides described herein comprise or consist of a modified oligonucleotide that optionally comprises a conjugate group. Conjugate groups may be attached to either or both ends of an oligonucleotide and/or at any internal position. In certain embodiments, conjugate groups are attached to the 2′-position of a nucleoside of a modified oligonucleotide. In certain embodiments, conjugate groups that are attached to either or both ends of an oligonucleotide are terminal groups. In certain such embodiments, conjugate moieties or terminal groups are attached at the 3′ and/or 5′-end of oligonucleotides. In certain such embodiments, conjugate moieties (or terminal groups) are attached at the 3′-end of oligonucleotides. In certain embodiments, conjugate moieties are attached near the 3′-end of oligonucleotides. In certain embodiments, conjugate moieties (or terminal groups) are attached at the 5′-end of oligonucleotides. In certain embodiments, conjugate moieties are attached near the 5′-end of oligonucleotides.

In certain embodiments, at least one internucleoside linkage has formula I:

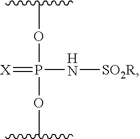

wherein R comprises a conjugate group. In certain embodiments, R is Ci₆.

### A. Certain Conjugate Groups and Conjugate Moieties

In certain embodiments, modified oligonucleotides comprise one or more conjugate moieties or conjugate groups. In certain embodiments, conjugate groups modify one or more properties of the molecule, including but not limited to pharmacodynamics, pharmacokinetics, stability, binding, absorption, tissue distribution, cellular distribution, cellular uptake, charge and clearance. In certain embodiments, conjugate moieties impart a new property on the molecule, e.g., fluorophores or reporter groups that enable detection of the molecule.

Certain conjugate groups have been described previously, for example: cholesterol moiety (Letsinger et al., Proc. Natl. Acad. Sci. USA, 1989, 86, 6553-6556), cholic acid (Manoharan et al., Bioorg. Med. Chem. Lett., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Lett., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic, a palmityl moiety (Mishra et al., Biochim. Biophys. Acta, 1995, 1264, 229-237), an octadecylamine or hexylamino-carbonyl-oxycholesterol moiety (Crooke et al., J Pharmacol. Exp. Ther., 1996, i, 923-937),_a tocopherol group (Nishina et al., Molecular Therapy Nucleic Acids, 2015, 4, e220; doi:10.1038/mtna.2014.72 and Nishina et al., Molecular Therapy, 2008, 16, 734-740), or a GalNAc cluster (e.g., WO2014/179620).

a. Conjugate Moieties

Conjugate moieties include, without limitation, intercalators, reporter molecules, polyamines, polyamides, peptides, carbohydrates (e.g., GalNAc), vitamin moieties, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins, fluorophores, and dyes.

In certain embodiments, a conjugate moiety comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, fingolimod, flufenamic acid, folinic acid, a benzothiadiazide, chlorothiazide, a diazepine, indo-methicin, a barbiturate, a cephalosporin, a sulfa drug, an antidiabetic, an antibacterial or an antibiotic.

b. Conjugate Linkers

In certain embodiments, conjugate groups comprise a conjugate linker that attaches a conjugate moiety to the remainder of the modified oligonucleotide. In certain embodiments, a conjugate linker is a single chemical bond (i.e. conjugate moiety is attached to the remainder of the modified oligonucleotide via a conjugate linker through a single bond). In certain embodiments, the conjugate linker comprises a chain structure, such as a hydrocarbyl chain, or an oligomer of repeating units such as ethylene glycol, nucleosides, or amino acid units.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described above, are bifunctional linking moieties, e.g., those known in the art to be useful for attaching conjugate groups to oligomeric compounds, such as the oligonucleotides provided herein. In general, a bifunctional linking moiety comprises at least two functional groups. One of the functional groups is selected to bind to a particular site on an oligomeric compound and the other is selected to bind to a conjugate group. Examples of functional groups used in a bifunctional linking moiety include but are not limited to electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

Examples of conjugate linkers include but are not limited to pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other conjugate linkers include but are not limited to substituted or unsubstituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In certain embodiments, conjugate linkers comprise 1-10 linker-nucleosides. In certain embodiments, such linker-nucleosides are modified nucleosides. In certain embodiments such linker-nucleosides comprise a modified sugar moiety. In certain embodiments, linker-nucleosides are unmodified. In certain embodiments, linker-nucleosides comprise an optionally protected heterocyclic base selected from a purine, substituted purine, pyrimidine or substituted pyrimidine. In certain embodiments, a cleavable moiety is a nucleoside selected from uracil, thymine, cytosine, 4-N-benzoylcytosine, 5-methylcytosine, 4-N-benzoyl-5-methylcytosine, adenine, 6-N-benzoyladenine, guanine and 2-N-isobutyrylguanine. It is typically desirable for linker-nucleosides to be cleaved from the oligomeric compound after it reaches a target tissue. Accordingly, linker-nucleosides are typically linked to one another and to the remainder of the oligomeric compound through cleavable bonds. In certain embodiments, such cleavable bonds are phosphodiester bonds. Unless otherwise indicated conjugate linkers comprise no more than 10 linker-nucleosides. In certain embodiments, conjugate linkers comprise no more than 5 linker-nucleosides. In certain embodiments, conjugate linkers comprise no more than 3 linker-nucleosides. In certain embodiments, conjugate linkers comprise no more than 2 linker-nucleosides. In certain embodiments, conjugate linkers comprise no more than 1 linker-nucleoside.

In certain embodiments, it is desirable for a conjugate group or conjugate moiety to be cleaved from the remainder of the oligonucleotide. For example, in certain circumstances oligomeric compounds (including oligomeric compounds that are antisense agents or portions thereof) or modified oligonucleotides comprising a particular conjugate moiety are better taken up by a particular cell type, but once the compound has been taken up, it is desirable that the conjugate group be cleaved to release an unconjugated oligonucleotide. Thus, certain conjugate moieties may comprise one or more cleavable moieties, typically within the conjugate linker. In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases.

In certain embodiments, a cleavable bond is selected from among: an amide, an ester, an ether, one or both esters of a phosphodiester, a phosphate ester, a carbamate, or a disulfide. In certain embodiments, a cleavable bond is one or both of the esters of a phosphodiester. In certain embodiments, a cleavable moiety comprises a phosphate or phosphodiester. In certain embodiments, the cleavable moiety is a phosphate or phosphodiester linkage between an oligonucleotide and a conjugate moiety or conjugate group.

In certain embodiments, a cleavable moiety comprises or consists of one or more linker-nucleosides. In certain such embodiments, one or more linker-nucleosides are linked to one another and/or to the remainder of the oligomeric compound through cleavable bonds. In certain embodiments, such cleavable bonds are unmodified phosphodiester bonds. In certain embodiments, a cleavable moiety is a nucleoside comprising a 2′-deoxyfuranosyl that is attached to either the 3′ or 5′-terminal nucleoside of an oligonucleotide by a phosphodiester internucleoside linkage and covalently attached to the remainder of the conjugate linker or conjugate moiety by a phosphodiester or phosphorothioate linkage. In certain such embodiments, the cleavable moiety is a nucleoside comprising a 2′-β-D-deoxyribosyl sugar moiety. In certain such embodiments, the cleavable moiety is 2′-deoxyadenosine.

c. Certain Cell-Targeting Conjugate Moieties

In certain embodiments, a conjugate group comprises a cell-targeting conjugate moiety. In certain embodiments, a conjugate group has the general formula:

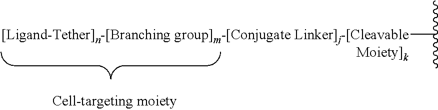

wherein n is from 1 to about 3, m is 0 when n is 1, m is 1 when n is 2 or greater, j is 1 or 0, and k is 1 or 0.

In certain embodiments, n is 1, j is 1 and k is 0. In certain embodiments, n is 1, j is 0 and k is 1. In certain embodiments, n is 1, j is 1 and k is 1. In certain embodiments, n is 2, j is 1 and k is 0. In certain embodiments, n is 2, j is 0 and k is 1. In certain embodiments, n is 2, j is 1 and k is 1. In certain embodiments, n is 3, j is 1 and k is 0. In certain embodiments, n is 3, j is 0 and k is 1. In certain embodiments, n is 3, j is 1 and k is 1.

In certain embodiments, conjugate groups comprise cell-targeting moieties that have at least one tethered ligand. In certain embodiments, cell-targeting moieties comprise two tethered ligands covalently attached to a branching group. In certain embodiments, cell-targeting moieties comprise three tethered ligands covalently attached to a branching group.

In certain embodiments, the cell-targeting moiety comprises a branching group comprising one or more groups selected from alkyl, amino, oxo, amide, disulfide, polyethylene glycol, ether, thioether and hydroxylamino groups. In certain embodiments, the branching group comprises a branched aliphatic group comprising groups selected from alkyl, amino, oxo, amide, disulfide, polyethylene glycol, ether, thioether and hydroxylamino groups. In certain such embodiments, the branched aliphatic group comprises groups selected from alkyl, amino, oxo, amide and ether groups.

In certain such embodiments, the branched aliphatic group comprises groups selected from alkyl, amino and ether groups. In certain such embodiments, the branched aliphatic group comprises groups selected from alkyl and ether groups. In certain embodiments, the branching group comprises a mono or polycyclic ring system.

In certain embodiments, each tether of a cell-targeting moiety comprises one or more groups selected from alkyl, substituted alkyl, ether, thioether, disulfide, amino, oxo, amide, phosphodiester, and polyethylene glycol, in any combination. In certain embodiments, each tether is a linear aliphatic group comprising one or more groups selected from alkyl, ether, thioether, disulfide, amino, oxo, amide, and polyethylene glycol, in any combination. In certain embodiments, each tether is a linear aliphatic group comprising one or more groups selected from alkyl, phosphodiester, ether, amino, oxo, and amide, in any combination. In certain embodiments, each tether is a linear aliphatic group comprising one or more groups selected from alkyl, ether, amino, oxo, and amid, in any combination. In certain embodiments, each tether is a linear aliphatic group comprising one or more groups selected from alkyl, amino, and oxo, in any combination. In certain embodiments, each tether is a linear aliphatic group comprising one or more groups selected from alkyl and oxo, in any combination. In certain embodiments, each tether is a linear aliphatic group comprising one or more groups selected from alkyl and phosphodiester, in any combination. In certain embodiments, each tether comprises at least one phosphorus linking group or neutral linking group. In certain embodiments, each tether comprises a chain from about 6 to about 20 atoms in length. In certain embodiments, each tether comprises a chain from about 10 to about 18 atoms in length. In certain embodiments, each tether comprises about 10 atoms in chain length.

In certain embodiments, each ligand of a cell-targeting moiety has an affinity for at least one type of receptor on a target cell. In certain embodiments, each ligand has an affinity for at least one type of receptor on the surface of a mammalian lung cell.

In certain embodiments, the cell-targeting moiety has affinity for the Asialoglycoprotein receptor (ASGPR). In certain embodiments, each ligand of a cell-targeting moiety is a carbohydrate, carbohydrate derivative, modified carbohydrate, polysaccharide, modified polysaccharide, or polysaccharide derivative. In certain such embodiments, the conjugate group comprises a carbohydrate cluster (see, e.g., Maier et al., “Synthesis of Antisense Oligonucleotides Conjugated to a Multivalent Carbohydrate Cluster for Cellular Targeting,” Bioconjugate Chemistry, 2003, 14, 18-29, or Rensen et al., “Design and Synthesis of Novel N-Acetylgalactosamine-Terminated Glycolipids for Targeting of Lipoproteins to the Hepatic Asiaglycoprotein Receptor,” J. Med. Chem. 2004, 47, 5798-5808, which are incorporated herein by reference in their entirety). In certain such embodiments, each ligand is an amino sugar or a thio sugar. For example, amino sugars may be selected from any number of compounds known in the art, such as sialic acid, α-D-galactosamine, β-muramic acid, 2-deoxy-2-methylamino-L-glucopyranose, 4,6-dideoxy-4-formamido-2,3-di-O-methyl-D-mannopyranose, 2-deoxy-2-sulfoamino-D-glucopyranose and N-sulfo-D-glucosamine, and N-glycolyl-a-neuraminic acid. For example, thio sugars may be selected from 5-Thio-β-D-glucopyranose, methyl 2,3,4-tri-O-acetyl-1-thio-6-O-trityl-α-D-glucopyranoside, 4-thio-β-D-galactopyranose, and ethyl 3,4,6,7-tetra-O-acetyl-2-deoxy-1,5-dithio-α-D-gluco-heptopyranoside.

In certain embodiments, oligomeric compounds (including oligomeric compounds that are antisense agents or portions thereof) or modified oligonucleotides described herein comprise a conjugate group found in any of the following references: Lee, Carbohydr Res, 1978, 67, 509-514; Connolly et al., J Biol Chem, 1982, 257, 939-945; Pavia et al., Int J Pep Protein Res, 1983, 22, 539-548; Lee et al., Biochem, 1984, 23, 4255-4261; Lee et al., Glycoconjugate J, 1987, 4, 317-328; Toyokuni et al., Tetrahedron Lett, 1990, 31, 2673-2676; Biessen et al., J Med Chem, 1995, 38, 1538-1546; Valentijn et al., Tetrahedron, 1997, 53, 759-770; Kim et al., Tetrahedron Lett, 1997, 38, 3487-3490; Lee et al., Bioconjug Chem, 1997, 8, 762-765; Kato et al., Glycobiol, 2001, 11, 821-829; Rensen et al., J Biol Chem, 2001, 276, 37577-37584; Lee et al., Methods Enzymol, 2003, 362, 38-43; Westerlind et al., Glycoconj J, 2004, 21, 227-241; Lee et al., BioorgMed Chem Lett, 2006, 16(19), 5132-5135; Maierhofer et al., BioorgMed Chem, 2007, 15, 7661-7676; Khorev et al., Bioorg Med Chem, 2008, 16, 5216-5231; Lee et al., Bioorg Med Chem, 2011, 19, 2494-2500; Kornilova et al., Analyt Biochem, 2012, 425, 43-46; Pujol et al., Angew Chemie IntEd Engl, 2012, 51, 7445-7448; Biessen et al., J Med Chem, 1995, 38, 1846-1852; Sliedregt et al., J Med Chem, 1999, 42, 609-618; Rensen et al., J Med Chem, 2004, 47, 5798-5808; Rensen et al., Arterioscler Thromb Vasc Biol, 2006, 26, 169-175; van Rossenberg et al., Gene Ther, 2004, 11, 457-464; Sato et al., JAm Chem Soc, 2004, 126, 14013-14022; Lee et al., J Org Chem, 2012, 77, 7564-7571; Biessen et al., FASEB J, 2000, 14, 1784-1792; Rajur et al., Bioconjug Chem, 1997, 8, 935-940; Duff et al., Methods Enzymol, 2000, 313, 297-321; Maier et al., Bioconjug Chem, 2003, 14, 18-29; Jayaprakash et al., Org Lett, 2010, 12, 5410-5413; Manoharan, Antisense Nucleic Acid Drug Dev, 2002, 12, 103-128; Merwin et al., Bioconjug Chem, 1994, 5, 612-620; Tomiya et al., Bioorg Med Chem, 2013, 21, 5275-5281; International applications WO1998/013381; WO2011/038356; WO1997/046098; WO2008/098788; WO2004/101619; WO2012/037254; WO2011/120053; WO2011/100131; WO2011/163121; WO2012/177947; WO2013/033230; WO2013/075035; WO2012/083185; WO2012/083046; WO2009/082607; WO2009/134487; WO2010/144740; WO2010/148013; WO1997/020563; WO2010/088537; WO2002/043771; WO2010/129709; WO2012/068187; WO2009/126933; WO2004/024757; WO2010/054406; WO2012/089352; WO2012/089602; WO2013/166121; WO2013/165816; U.S. Pat. Nos. 4,751,219; 8,552,163; 6,908,903; 7,262,177; 5,994,517; 6,300,319; 8,106,022; 7,491,805; 7,491,805; 7,582,744; 8,137,695; 6,383,812; 6,525,031; 6,660,720; 7,723,509; 8,541,548; 8,344,125; 8,313,772; 8,349,308; 8,450,467; 8,501,930; 8,158,601; 7,262,177; 6,906,182; 6,620,916; 8,435,491; 8,404,862; 7,851,615; Published U.S. Patent Application Publications US2011/0097264; US2011/0097265; US2013/0004427; US2005/0164235; US2006/0148740; US2008/0281044; US2010/0240730; US2003/0119724; US2006/0183886; US2008/0206869; US2011/0269814; US2009/0286973; US2011/0207799; US2012/0136042; US2012/0165393; US2008/0281041; US2009/0203135; US2012/0035115; US2012/0095075; US2012/0101148; US2012/0128760; US2012/0157509; US2012/0230938; US2013/0109817; US2013/0121954; US2013/0178512; US2013/0236968; US2011/0123520; US2003/0077829; US2008/0108801; and US2009/0203132.

In certain embodiments, the conjugate group comprises N-acetylgalactosamine (GalNAc).

In certain embodiments, the conjugate group is attached to the first modified oligonucleotide at the 5′-end of the first modified oligonucleotide. In certain embodiments, the conjugate group is attached to the first modified oligonucleotide at the 3′-end of the modified oligonucleotide.

In certain embodiments, the conjugate group comprises a cell-targeting moiety having an affinity for transferrin receptor (TfR), also known as TfR1 and CD71. In certain embodiments, the conjugate group comprises an anti-TfR1 antibody or fragment thereof. In certain embodiments, the conjugate group comprises a peptide capable of binding TfR1. In certain embodiments, the conjugate group comprises an aptamer capable of binding TfR1.

### Compositions and Methods for Formulating Pharmaceutical Compositions

Antisense agents, oligomeric compounds, and modified oligonucleotides described herein may be admixed with pharmaceutically acceptable active or inert substances for the preparation of pharmaceutical compositions. Compositions and methods for the formulation of pharmaceutical compositions are dependent upon a number of criteria, including, but not limited to, route of administration, extent of disease, or dose to be administered.

Certain embodiments provide pharmaceutical compositions comprising one or more oligomeric compounds (including oligomeric compounds that are antisense agents or portions thereof) or a salt thereof. In certain such embodiments, the pharmaceutical composition comprises a suitable pharmaceutically acceptable diluent or carrier. In certain embodiments, a pharmaceutical composition comprises a sterile saline solution and one or more oligomeric compound. In certain embodiments, such pharmaceutical composition consists of a sterile saline solution and one or more oligomeric compound. In certain embodiments, the sterile saline is pharmaceutical grade saline. In certain embodiments, a pharmaceutical composition comprises one or more oligomeric compound and sterile water. In certain embodiments, a pharmaceutical composition consists of one oligomeric compound and sterile water. In certain embodiments, the sterile water is pharmaceutical grade water. In certain embodiments, a pharmaceutical composition comprises or consists of one or more oligomeric compound and phosphate-buffered saline (PBS). In certain embodiments, a pharmaceutical composition consists of one or more oligomeric compound and sterile PBS. In certain embodiments, the sterile PBS is pharmaceutical grade PBS. Compositions and methods for the formulation of pharmaceutical compositions are dependent upon a number of criteria, including, but not limited to, route of administration, extent of disease, or dose to be administered.

An oligomeric compound described herein complementary to a target nucleic acid can be utilized in pharmaceutical compositions by combining the oligomeric compound with a suitable pharmaceutically acceptable diluent or carrier and/or additional components such that the pharmaceutical composition is suitable for injection. In certain embodiments, a pharmaceutically acceptable diluent is phosphate buffered saline. Accordingly, in one embodiment, employed in the methods described herein is a pharmaceutical composition comprising an oligomeric compound complementary to a target nucleic acid and a pharmaceutically acceptable diluent. In certain embodiments, the pharmaceutically acceptable diluent is phosphate buffered saline. In certain embodiments, the oligomeric compound comprises or consists of a modified oligonucleotide provided herein.

Pharmaceutical compositions comprising oligomeric compounds (including oligomeric compounds that are antisense agents or portions thereof) provided herein encompass any pharmaceutically acceptable salts, esters, or salts of such esters, or any other oligonucleotide which, upon administration to an animal, including a human, is capable of providing (directly or indirectly) the biologically active metabolite or residue thereof. In certain embodiments, the oligomeric compound comprises or consists of a modified oligonucleotide. Accordingly, for example, the disclosure is also drawn to pharmaceutically acceptable salts of compounds, prodrugs, pharmaceutically acceptable salts of such prodrugs, and other bioequivalents. Suitable pharmaceutically acceptable salts include, but are not limited to, sodium and potassium salts.

### Target Nucleic Acids, Target Regions and Nucleotide Sequences

In certain embodiments, antisense agents, oligomeric compounds, or modified oligonucleotides described herein comprise or consist of an oligonucleotide comprising a region that is complementary to a target nucleic acid. In certain embodiments, the target nucleic acid is an endogenous RNA molecule. In certain embodiments, the target nucleic acid encodes a protein. In certain such embodiments, the target nucleic acid is selected from: an mRNA and a pre-mRNA, including intronic, exonic and untranslated regions. In certain embodiments, the target RNA is an mRNA. In certain embodiments, the target nucleic acid is a pre-mRNA. In certain embodiments, a pre-mRNA and corresponding mRNA are both target nucleic acids of a single compound. In certain such embodiments, the target region is entirely within an intron of a target pre-mRNA. In certain embodiments, the target region spans an intron/exon junction. In certain embodiments, the target region is at least 50% within an intron. In certain embodiments, the target nucleic acid is a microRNA. In certain embodiments, the target region is in the 5′ UTR of a gene. In certain embodiments, the target region is within a translation suppression element region of a target nucleic acid.

### Certain Compounds

Certain compounds described herein (e.g., antisense agents, oligomeric compounds, and modified oligonucleotides) have one or more asymmetric center and thus give rise to enantiomers, diastereomers, and other stereoisomeric configurations that may be defined, in terms of absolute stereochemistry, as (R) or (S), as a or β such as for sugar anomers, or as (D) or (L), such as for amino acids, etc. Compounds provided herein that are drawn or described as having certain stereoisomeric configurations include only the indicated compounds. Compounds provided herein that are drawn or described with undefined stereochemistry include all such possible isomers, including their stereorandom and optically pure forms. All tautomeric forms of the compounds provided herein are included unless otherwise indicated.

The compounds described herein include variations in which one or more atoms are replaced with a non-radioactive isotope or radioactive isotope of the indicated element. For example, compounds herein that comprise hydrogen atoms encompass all possible deuterium substitutions for each of the ¹H hydrogen atoms. Isotopic substitutions encompassed by the compounds herein include but are not limited to: ²H or ³H in place of ¹H, ¹³C or ¹⁴C in place of ¹²C, ¹⁵N in place of ¹⁴N, ¹⁷O or ¹⁸O in place of ¹⁶O, and ³³S, ³⁴S, ³⁵S, or ³⁶S in place of ³²S. In certain embodiments, non-radioactive isotopic substitutions may impart new properties on the oligomeric compound that are beneficial for use as a therapeutic or research tool. In certain embodiments, radioactive isotopic substitutions may make the compound suitable for research or diagnostic purposes such as imaging.

### EXAMPLES

The following examples are intended to illustrate certain aspects of the invention and are not intended to limit the invention in any way.

### Example 1: Preparation of Oxidizing Solutions

Five oxidizing solutions each comprising sulfonyl oxidizing agent methanesulfonyl azide (mesyl azide, MsN₃) were prepared. One oxidizing solution contained no stabilizing agent (control solution), and four oxidizing solutions each included a stabilizing agent. The stabilizing agents tested were triphenyl phosphate (TPP) and diphenyl sulfone (DPS):

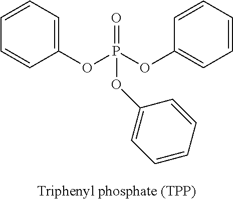 [SMILES: O=P(Oc1ccccc1)(Oc1ccccc1)Oc1ccccc1]

Use of diphenyl sulfone is not recommended, as crystalline material was observed to form.

The solutions were prepared as described below and with concentrations specified in Table 1. All solutions were stored at 5° C.

TABLE 1
Oxidizing solutions

| | Sulfonyl | | |
|---|---|---|---|
| | Oxidizing | | |
| Oxidizing | Agent | Stabilizing | |
| Solution | MSN₃ (M) | Agent | Solvents |
| 1 | 0.5 | none | 1:1 acetonitrile to toluene |
| 2 | 0.5 | TPP, 1.0M | 1:1 acetonitrile to toluene |

### Preparation of 1.0 MsN₃ in Acetonitrile

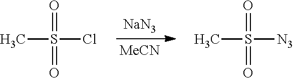

NaN₃ (5.4 g, 83 mmol, 1 eq) was suspended in dry acetonitrile (MeCN, 80 mL), and was cooled to 0° C. while stirring under nitrogen. Mesyl chloride (10 g, 6.75 ml, 88.3 mmol, 1.05 eq.) was added dropwise, and the reaction was allowed to warm to room temperature over the course of 3 hours. The reaction was filtered to remove insoluble salts, resulting in the desired 1.0 M MsN₃ solution in MeCN.

### Preparation of Oxidizing Solution 1

20 mL of toluene and 20 mL of 1.0 M MsN₃ in MeCN (described above) were added to a 50 mL amber bottle. The bottle was covered with parafilm and capped. The solution was mixed by hand swirling.

### Preparation of Oxidizing Solution 2

10 mL of 2.0 M TPP in toluene and 10 mL of 1.0 M MsN₃ in MeCN (described above) were added to a 50 mL amber bottle. The bottle was covered with parafilm and capped. The solution was mixed by hand swirling.

### Example 2: Thermodynamic Analysis of Oxidizing Solutions

Oxidizing solutions 1-3 described above were tested by Dekra (dekra.us/process-safety) using their standard protocol Briefly, solvent was removed from each oxidizing solution, and the corresponding residues were evaluated by differential scanning calorimetry (DSC) to determine their heats of composition. This test determines the onset temperature of any energetic events and the total energy associated with those events. Each event is described in a separate row in Table 2 below and labeled for whether there was a positive (endotherm) or negative (exotherm) change in enthalpy of the residue as the residue is heated. An energy of decomposition greater than 300 J·g⁻¹ indicates a highly energetic material, and an energy of decomposition greater than 500 J·g⁻¹ suggests the material may have explosive properties.

A 0.400 mL portion of each solution was evaporated overnight at room temperature, producing a residue which was used in DSC testing. The test sample was charged to a sealed high-pressure gold crucible. An empty crucible of the same type was used as a reference. The sample and reference crucibles were placed into the Mettler Toledo DSC 3+ furnace which was heated to the start temperature of 25° C. Once the crucibles equilibrated with the furnace, they were heated at a constant rate of 5° C./min up to 400° C. The heat flow from the sample crucible and the reference crucible were recorded throughout the test. Any exothermic activity within the sample will result in a larger heat flow out of the sample crucible relative to the reference crucible. Start temperature, end temperature, and peak temperature were recorded and are provided in Table 2.

TABLE 2
DSC results of Oxidizing solutions

| | | Start | Peak | End | |
|---|---|---|---|---|---|
| | | Temper- | Temper- | Temper- | |
| Residue | | ature | ature | ature | Energy |
| Source | Event | (° C.) | (° C.) | (° C.) | (J g⁻¹) |
| Oxidizing | Exotherm | 100.8 | 185.0 | 218.4 | 2083.12 |
| solution 1 | Exotherm | 218.7 | 240.4 | 255.0 | 146.14 |
| | Exotherm | 297.0 | 352.7 | 371.9 | 66.63 |
| Oxidizing | Exotherm | 142.0 | 192.8 | 219.1 | 259.32 |
| solution 2 | Exotherm | 234.0 | 295.4 | 326.2 | 117.09 |

As shown above, residues containing TPP (oxidizing solution 2) have lower combustion energies than the residue from the control solution without any solid stabilizing agent (oxidizing solution 1).

### Example 3: Synthesis of Modified Oligonucleotides Containing Mesyl Phosphoramidate Internucleoside Linkages Via Oxidative Mesylation Using Mesyl Azide Solutions

The following figure shows a general, high-level scheme of oligonucleotide synthesis using mesyl azide as an oxidizing agent. An oligonucleotide intermediate, displayed as a black and white ribbon, is attached onto a solid support (shown as a circle). A phosphoramidite monomer is incorporated onto the oligonucleotide using standard techniques. “B(pg)” in the figure below represents a variable nucleobase with generic protecting group(s). Use of mesyl azide as the oxidizing agent in the second general step results in oxidation of the phosphotriester linkage to yield a mesyl phosphoramidate internucleoside linkage.

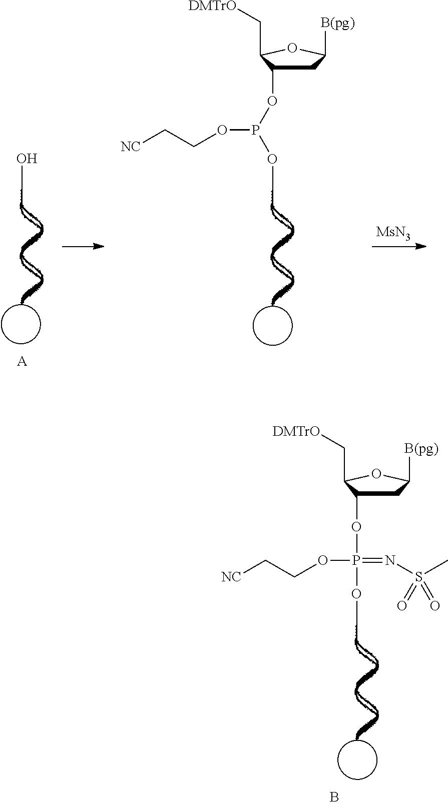

Modified oligonucleotides comprising mesyl phosphoramidate internucleoside linkages were prepared using the oxidizing solutions described in Example 1.

The oligonucleotide intermediate, Compound A, was synthesized using standard techniques. Compound A is a modified oligonucleotide intermediate 13 nucleosides in length having nucleobase sequence (from 5′ to 3′): TGGTTATGACTCA (SEQ ID NO: 1). The sugar motif of Compound A is (from 5′ to 3′): ddddddddeeeee; wherein each “d” represents a 2′-β-D-deoxyribosyl sugar moiety, and each “e” represents a 2′-MOE sugar moiety. The internucleoside linkage motif of Compound A is (from 5′ to 3′): ssssssssssss, wherein each “s” represents a phosphorothioate internucleoside linkage. Each cytosine residue is a 5-methylcytosine. The 5′-OH of Compound A is capped with a dimethoxytrityl (DMT) protecting group. The linked nucleosides of Compound A are attached to a solid support.

Two 2′-β-D-deoxyribosyl thymidine nucleosides were joined to Compound A via mesyl phosphoramidate internucleoside linkages. Modified oligonucleotides were synthesized on an AKTA Oligopilot 10 (35 μmol scale). Deoxy T phosphoramidite was dissolved in 1:1 MeCN/toluene (v/v) and was dried over molecular sieves. The DMT protecting group was removed from the modified oligonucleotide intermediate using 15% DCA in toluene. Deoxy T phosphoramidite was coupled using 3 equivalents of amidite and 10 equivalents of activator (1 M 4,5-dicyanoimidazole and 0.1 M N-methylimidazole in acetonitrile) relative to amidite, and the coupling solution was allowed to recycle for 6 minutes. After flushing with MeCN, the oxidizing solution comprising MsN₃ was added (25 equiv) and was allowed to recycle for 25 minutes followed by an MeCN wash. After washing, the reaction mixture was treated with 20% acetic anhydride in MeCN (Cap A) and N-methylimidazole in MeCN/pyridine (2:5:3 v/v/v, Cap B) to cap any coupling failures. The cycle was repeated to incorporate the second mesyl-linked thymidine nucleoside.

The modified oligonucleotide intermediate, Compound B, was cleaved from solid support and deprotected using standard techniques to yield the final modified oligonucleotide. Compound B is a modified oligonucleotide 15 nucleosides in length having sequence (from 5′ to 3′): TTTGGTTATGACTCA (SEQ ID NO: 2). The sugar motif of Compound B is (from 5′ to 3′): ddddddddddeeeee; wherein each “d” represents a 2′-β-D-deoxyribosyl sugar moiety, and each “e” represents a 2′-MOE sugar moiety. The internucleoside linkage motif of Compound B is (from 5′ to 3′): zzssssssssssss, wherein each “s” represents a phosphorothioate internucleoside linkage, and each “z” represents a mesyl phosphoramidate internucleoside linkage. Each cytosine residue is a 5-methylcytosine.

Oxidizing solutions 1-5 were each used to synthesize Compound B separately, resulting in products analogously labeled Compounds B1, B2, B3, B4, and B5. Compounds B1-B5 were analyzed by UV chromatography and liquid chromatography-mass spectrometry. In the oxidative mesylation step, oxidizing solution 2 (TPP in 1:1 toluene to MeCN) led to reaction completion and acceptable purity compared to Compound B1 (synthesized using the control solution). In oxidizing solutions 2, 3, and 4, the stabilizing agents appeared to have no deleterious effect on coupling and may reduce hazardous risk of working with mesyl azide.

### Example 4: Preparation of Methanesulfonyl Azide Solution in Acetonitrile with Sulfolane as a Stabilizing Agent

An oxidizing solution comprising sulfonyl oxidizing agent MsN₃ in acetonitrile, including sulfolane as a stabilizing agent was prepared. The structure of sulfolane is shown below:

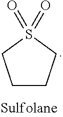

The solution was prepared using two methods, as described below.

### Method 1

A 3-neck 1000 mL round bottom flask with overhead stirrer (Teflon moon-shaped impellor, glass shaft) under N₂ was charged with NaN₃ (70.58 g, 1.09 mol), and dry MeCN (517 mL). Stirring began at room temperature at a setting of ca. 2.5 on the IKA overhead stirrer for ca. 5 minutes before the flask was submerged in an ice bath. Mesyl chloride (80.0 mL, 1.03 mol) was added dropwise via addition funnel over ca. 30 minutes. Upon complete addition of the mesyl chloride, the reaction was removed from the ice bath and allowed to stir at room temperature overnight.

The reaction was confirmed complete based on the absence of the chemical shift for the mesyl chloride (expected: 3.8 ppm) by ¹H NMR. The concentration of MsN₃ was determined to be 2.082 M by quantitative NMR using ethylene carbonate as the analytical standard.

The reaction was then filtered through a bottle-top filter into a tared polycoated glass bottle with Teflon coated-magnetic stir bar. The filter cake was rinsed with a small volume of MeCN (˜10 mL). The bottle was then charged with melted sulfolane (621.28 g, 5.17 mol). The mixture was stirred, and the solution mass and density were determined and used to dilute the solution with MeCN to a total volume of 1034 mL.

### Method 2

A 3-neck 1000 mL round bottom flask with overhead stirrer (Teflon moon-shaped impellor, glass shaft) under N₂ was charged with NaN₃ (35.29 g, 543 mmol), melted sulfolane (246 g, 2.59 mol) and dry MeCN (270 mL). Stirring began at room temperature at a setting of ca. 2.5 on the IKA overhead stirrer for ca. 5 minutes before the flask was submerged in an ice bath. Mesyl chloride (40.0 mL, 517 mmol) was added dropwise via addition funnel over ca. 30 minutes. Upon complete addition of the Mesyl chloride, the reaction was removed from the ice bath and allowed to stir at room temperature overnight.

The reaction was confirmed complete based on the absence of the chemical shift for the Mesyl chloride (expected: 3.8 ppm) by ¹H NMR. The concentration of MsN₃ was determined to be 0.943 M in the reaction mixture by quantitative NMR using ethylene carbonate as the analytical standard.

The reaction was then filtered through a bottle-top filter into a tared polycoated glass bottle and stored without further dilution.

### Example 5: Synthesis of Modified Oligonucleotides Containing Mesyl Phosphoramidate Internucleoside Linkages Via Oxidative Mesylation Using Mesyl Azide Solutions

Modified oligonucleotides comprising mesyl phosphoramidate internucleoside linkages were prepared using a solution of MsN₃ in acetonitrile with sulfolane.

Modified oligonucleotides were synthesized on an AKTA Oligopilot 10 (40 μmol scale) using polystyrene based NittoPhase HL UnyLinker support (405 μmol/g). Fully protected nucleoside phosphoramidites were incorporated using standard solid-phase modified oligonucleotide synthesis conditions, described herein above in Example 3. DNA amidites were dissolved at 0.1 M in 1:1 MeCN/toluene and incorporated using 6 min recycling times. 1 M 4,5-dicyanoimidazole with 0.1 M N-methylimidazole in MeCN was used as an activator. DMT protecting group were removed using 15% dichloroacetic acid in toluene. 20% acetic anhydride in MeCN and N-methylimidazole/pyridine/MeCN (20:30:50) was used for capping coupling failures.

Oxidations of the P(III) species were performed as follows: 0.05 M iodine in pyridine/H₂O (9:1) for phosphodiester linkages; or 0.1 M xanthane hydride in 1:1 pyridine:MeCN for phosphorothioate linkages. For incorporation of mesylphosphoramidate linkages, the modified oligonucleotide intermediate was treated with 0.65 M MsN₃ in 1:1 MeCN:sulfolane or 0.65 M MsN₃ in MeCN and allowed to recycle for 25 minutes.

After conclusion of the synthesis, the cyanoethyl protecting groups were removed using 20% diethylamine in toluene, and the remaining protecting groups were cleaved by suspending the solid support in aqueous concentrated ammonia and heating at 55° C. for 14 h. The support was removed by filtration and the crude mixture was purified by HPLC using a combined purification, detritylation, desalt method. During the basic RSR (Reverse phase, SAX, Reverse phase) method the sample was loaded onto the RP column (DuPont XT30) in H₂O. A failure elution was then performed on the RP column with 1:1 (A: 80% MeOH/water, B: 2.5 M NaCl, 50 mM NaOH). DMT cleavage was then performed on the RP column with 6% DCA, followed by a water wash. Next, the detritylated compound was loaded onto the SAX column with 80% MeOH. The RP column was equilibrated with 50 mM NaOH. A SAX gradient was then performed from 0 to 50% with A and B buffers (A: 50 mM NaOH, B: 50 mM NaOH, 2.5 M NaCl). Once a UV absorbance threshold was reached, the compound was then loaded back onto the RP column. NaCl (250 mM) was flushed through the RP column for cation exchange, water was flowed through the column for desalt, and the final compound was eluted in 1:1 MeCN:water.

Comparison of Product Purity after Synthesis with and without Sulfolane

Modified oligonucleotide Compound 1633475 was synthesized using standard techniques described herein above, with and without sulfolane, and each lot was analysed for lot purity.

Compound 1633475 is a modified oligonucleotide intermediate 16 nucleosides in length having nucleobase sequence (from 5′ to 3′): GCATGTTCTCACATTA (SEQ ID NO: 3). The sugar motif of Compound 1633475 is (from 5′ to 3′): kkkddddddddddkkk; wherein each “d” represents a 2′-β-D-deoxyribosyl sugar moiety, and each “k” represents a cEt sugar moiety. The internucleoside linkage motif of Compound 1633475 is (from 5′ to 3′): ssszzzzssssssss, wherein each “s” represents a phosphorothioate internucleoside linkage, and each “z” represents a mesyl phosphoramidate internucleoside linkage. Each cytosine residue is a 5-methylcytosine. Compound 1633475 further contains a 3THAGNhp moiety conjugated to the 3′-terminal oxygen of the modified oligonucleotide via a phosphodiester bond as shown below:

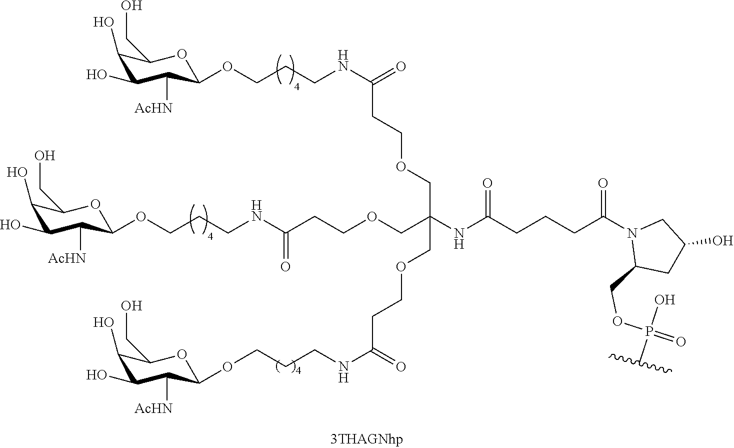

A sample of each modified oligonucleotide lot was made in 0.01% triethylamine in H₂O at an approximate concentration of 1 mg/mL. The samples were analyzed by Ion Pair HPLC/mass spectrometry (IP-HPLC/MS) on an Agilent 1200 series equipped with a binary pump, online degasser, heated column chamber, autosampler, and multiple wavelength UV detector, interfaced to an electrospray mass spectrometer. Analysis was performed using a Waters (Milford, MA, USA) XBridge™ HPLC column (18C, 3.5 μm, 2.1×150 mm, Waters P/N 186003023). A linear gradient of 5 mM tributylammonium acetate with 1 μM EDTA in 10% acetonitrile (Mobile Phase A) and 5 mM tributylammonium acetate with 1 μM EDTA in 80% acetonitrile (Mobile Phase B) was used, as described in the table below.

TABLE 3
HPLC linear gradient, flow rate: 0.25 mL/min

| Time (min) | Mobile Phase A (%) | Mobile Phase B (%) |
|---|---|---|
| 0 | 55 | 45 |
| 22 | 20 | 80 |
| 25 | 20 | 80 |
| 26 | 55 | 45 |

The UV absorbance of column eluate was measured at 260 nm, using a reference wavelength of 400 nm. Column eluate was introduced directly into the ESI-MS. The ESI source was operated in negative mode, with a scanning mass signal (m/z) range of (full length product mass)/4+150.0. Capillary voltage=4000 V; drying gas temperature=260° C.; drying gas flow=12 L/min; nebulizer pressure=25 psi; fragmentor voltage=100 V.

To calculate UV purity, the full-length product (main UV peak), early eluting impurity, and late eluting impurity UV peaks at 260 nm were identified and integrated in OpenLab ChemStation version C.01.09. The area of the main UV peak was normalized to the sum area of all peaks at 260 nm and is presented in the tables below as UV Purity (%). To calculate MS purity, the full-length product m/z and all impurity m/z were identified within the main UV peak. The ion chromatogram for each component mass signal was extracted and integrated. The area of the full-length product signal was normalized to the sum of the component signals and is presented below as MS purity (%). Each table represents a different analysis.

Synthesis of Compound 1633475 using 0.65 M MsN₃ in MeCN with or without 1.5 M sulfolane produced modified oligonucleotide of similar quality. UV and MS purity is provided in Table 4. The synthesis conditions used were the same with the exception of the solvent carrying the MsN₃ oxidizing solution.

TABLE 4
Purity comparison of Compound 1633475 synthesized
with and without 1.5M sulfolane, analyzed by
UV and mass spectrometry (average of two lots)

| Analysis | No Sulfolane | 1.5M Sulfolane |
|---|---|---|
| UV Purity (%) | 88 | 87 |
| MS Purity (%) | 76 | 79 |

### Example 6: Thermodynamic Analysis of Mesyl Azide in the Presence of Sulfolane

An oxidizing solution MsN₃ stabilized by sulfolane was tested by Nalas Engineering (nalasengineering.com/process-scale-up) using a standard DSC protocol.

A portion of each solution was evaporated overnight at room temperature, producing a residue which was used in DSC testing. Analysis was performed using a DSC 25 (Waters Instruments). The test sample was charged to a sealed high-pressure gold crucible. An empty crucible of the same type was used as a reference. The sample and reference crucibles were placed into the furnace which was heated to the start temperature of 30° C. Once the crucibles equilibrated with the furnace, they were heated at a constant rate of 5° C./min up to 500° C. The heat flow from the sample crucible and the reference crucible were recorded throughout the test and analyzed using TRIOS software. The results are presented in the table below as Energy Flow, with the event presented as exotherm (negative change in enthalpy) or endotherm (positive change in enthalpy). An energy of decomposition greater than 300 J g⁻¹ indicates a highly energetic material, and an energy of decomposition greater than 500 J g⁻¹ suggests the material may have explosive properties. As shown in the table below, the residue containing sulfolane has a lower combustion energy than neat MsN₃.

TABLE 5
DSC results of MsN₃ stabilized by sulfolane

| | | Energy Flow | Start Temperature |
|---|---|---|---|
| Material | Event | (J g⁻¹) | (° C.) |
| Neat MsN₃ | Exotherm | 2083 | 100.8° C. |
| 1.1M MsN₃, | Exotherm | 463 | 138.4° C. |
| 4.4M Sulfolane | | | |
| solution in MeCN | | | |

### Example 7: Impact Sensitivity Analysis of Mesyl Azide in the Presence of Sulfolane

An oxidizing solution of MsN₃, stabilized by sulfolane was tested by Nalas Engineering (nalasengineering.com/process-scale-up) using a standard protocol for impact sensitivity. Briefly, a sample of material was placed in a BAM Fall Hammer Apparatus and impacted with various amounts of energy. The sample was observed for flash, flame, or explosion to determine the limiting impact energy. Observations for MsN₃ with sulfolane, and neat MsN₃ are presented in the table below.

By impact test, ˜1:1 (w/w) mixture of MsN₃:sulfolane appears to mitigate the explosive properties of MsN₃.

TABLE 6
Results of Impact testing on MsN₃ in the presence of sulfolane

| | Mesyl Azide | Impact Energy | |
|---|---|---|---|
| Material | (wt %) | (J) | Observations |
| Neat MsN₃ | >99.0 | <1 | Explosion |
| | | | (flash, smoke, odor) |
| MsN₃/sulfolane | 46.5 | 2.5 | Mild reaction at all |
| | | | levels, decomposition |
| | | | (odor) at 5 J and 10 J |

What is claimed:

1. A method of preparing a modified oligonucleotide comprising contacting a first oligonucleotide intermediate having a phosphite triester internucleoside linkage with at least one stabilizing agent and with an oxidizing solution comprising a sulfonyl oxidizing agent to form a second oligonucleotide intermediate having an internucleoside linking group of Formula XIV: 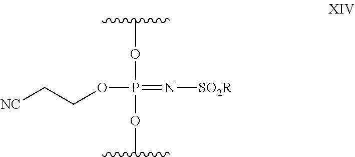
wherein:
R is selected from aryl, a substituted aryl, a heterocycle, a substituted heterocycle, an aromatic heterocycle, a substituted aromatic heterocycle, a diazole, a substituted diazole, a C₁-C₆ alkoxy, C₁-C₂₀ alkyl, C₁-C₆ alkenyl, C₁-C₆ alkynyl, substituted C₁-C₂₀ alkyl, substituted C₁-C₆ alkenyl substituted C₁-C₆ alkynyl, and a conjugate group.

2. The method of claim 1, wherein R is methyl and the sulfonyl oxidizing agent is methanesulfonyl azide (MsN₃).

3. The method of any of claims 1 to 2, wherein the oxidizing solution comprises the at least one stabilizing agent.

4. The method of any of claims 1 to 2, wherein the oxidizing solution does not comprise the at least one stabilizing agent.

5. The method of any of claims 1 to 4, wherein the oxidizing solution comprises a solvent selected from acetonitrile, toluene, dichloromethane, pyridine, N-methyl-2-pyrrolidone, and combinations thereof.

6. The method of any of claims 1 to 5, wherein the at least one stabilizing agent is selected from sulfolane and triphenylphosphate (TPP).

7. The method of any of claims 1 to 6, wherein at least one stabilizing agent is sulfolane, optionally wherein sulfolane is sole stabilizing agent.

8. The method of any of claims 1 to 7, wherein at least one stabilizing agent is TPP, optionally wherein TPP is sole stabilizing agent.

9. The method of any of claims 1 to 8, wherein at least one stabilizing agent is a non-crosslinked polymer.

10. The method of claim 9, wherein the non-crosslinked polymer is polystyrene.

11. The method of any of claims 1 to 10, wherein a residue obtained by evaporating the solvent from the oxidizing solution and the at least one stabilizing agent has combustion energy less than 500 J·g⁻¹.

12. The method of any of claims 1 to 11, wherein residue obtained by evaporating the solvent from the oxidizing solution and the at least one stabilizing agent has combustion energy less than 300 J·g⁻¹.

13. The method of any preceding claim, wherein the oxidizing solution comprises 0.1 to 10 equivalents of the at least one stabilizing agent relative to the sulfonyl oxidizing agent.

14. The method of claim 13, wherein the oxidizing solution comprises 1 to 10 equivalents of the at least one stabilizing agent relative to the sulfonyl oxidizing agent.

15. The method of claim 13, wherein the oxidizing solution comprises 3 to 6 equivalents of the at least one stabilizing agent relative to the sulfonyl oxidizing agent.

16. The method of claim 13, wherein the oxidizing solution comprises 4 to 5 equivalents of the at least one stabilizing agent relative to the sulfonyl oxidizing agent.

17. The method of any of the preceding claims, wherein the oxidizing solution comprises 0.1 to 10 M of the sulfonyl oxidizing agent.

18. The method of any of the preceding claims, wherein the oxidizing solution comprises 0.5 to 5 M of the sulfonyl oxidizing agent.

19. The method of any of the preceding claims, wherein the oxidizing solution comprises 0.6 to 1.5 M of the sulfonyl oxidizing agent.

20. A method of synthesizing a modified oligonucleotide comprising at least one internucleoside linkage of Formula I: 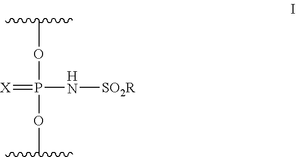
wherein independently for each internucleoside linkage of Formula I:
X is selected from O and S, and
R is selected from aryl, a substituted aryl, a heterocycle, a substituted heterocycle, an aromatic heterocycle, a substituted aromatic heterocycle, a diazole, a substituted diazole, a C₁-C₆ alkoxy, C₁-C₂₀ alkyl, C₁-C₆ alkenyl, C₁-C₆ alkynyl, substituted C₁-C₂₀ alkyl, substituted C₁-C₆ alkenyl substituted C₁-C₆ alkynyl, and a conjugate group;
wherein the method comprises steps of:
a) providing a solid support having a first blocked hydroxyl group attached thereto;
b) adding a deblocking agent to the reaction to deblock the first blocked hydroxyl group to provide a free first hydroxyl group;
c) adding a nucleoside to the reaction to couple at the free first hydroxyl group, wherein the nucleoside comprises a phosphoramidite group and a second blocked hydroxyl group, to provide phosphite triester linked nucleosides;
d) adding to the reaction:
1. a standard oxidizing agent to produce a phosphate triester internucleoside linkage;
2. a standard sulfurizing agent to produce a thiophosphate triester internucleoside linkage; or
3. a sulfonyl oxidizing agent and at least one stabilizing agent to produce a sulfonyl phosphoramidate internucleoside linkage;
e) optionally treating the sufonyl phosphoramidate, phosphate triester, or thiophosphate triester linkages with a capping reagent to cap any unreacted free hydroxyl groups;
f) iteratively repeating steps b) through e) a predetermined number of times to provide the modified oligonucleotide, provided that at least one iteration includes step (d)3;
g) treating the modified oligonucleotide with triethylamine or diethylamine in acetonitrile; and
h) optionally treating the modified oligonucleotide with ammonium hydroxide to cleave the modified oligonucleotide from the solid support;
thereby synthesizing the modified oligonucleotide comprising at least one internucleoside linkage of Formula I.

21. The method of claim 20, wherein each R is methyl.

22. The method of claim 20 or 21, wherein the sulfonyl oxidizing agent is methanesulfonyl azide and the oxidizing solution comprises the methanesulfonyl azide and the at least one stabilizing agent.

23. The method of any one of claims 20-22, wherein the at least one stabilizing agent is sulfolane, optionally wherein sulfolane is sole stabilizing agent.

24. The method of any one of claims 20-22, wherein the at least one stabilizing agent is TPP, optionally wherein TPP is sole stabilizing agent.

25. The method of any of claims 20 to 24, wherein a residue obtained by evaporation of the solvent from the solution comprising the sulfonyl oxidizing agent and the at least one stabilizing agent has combustion energy less than 500 J·g⁻¹.

26. The method of any of claims 20 to 24, wherein a residue obtained by evaporation of the solvent from the solution comprising the sulfonyl oxidizing agent and the at least one stabilizing agent has combustion energy less than 300 J·g¹.

27. The method of any of claims 20 to 26, wherein each X is O.

28. The method of any one of claims 20-27, wherein the capping reagent is acetic anhydride.

29. The method of any one of claims 20-28, comprising treating the modified oligonucleotide with ammonium hydroxide to remove protecting groups and cleave the modified oligonucleotide from the solid support.

30. The method of any one of claims 20-29, wherein the modified oligonucleotide comprises 12 to 25 linked nucleosides.

31. The method of any one of claims 1-30, wherein the modified oligonucleotide comprises phosphorothioate and mesyl phosphoramidate, and optionally phosphodiester internucleoside linkage(s).

32. The method of any one of claims 1-30, wherein the modified oligonucleotide comprises internucleoside linkages selected from phosphodiester, phosphorothioate, and mesyl phosphoramidate internucleoside linkage(s), and no other internucleoside linkages.

33. The method of any one of claims 1-32, wherein the modified oligonucleotide comprises a stereo-standard sugar moiety, a cEt sugar moiety, a 2′-MOE sugar moiety, a 2′-OMe sugar moiety, a 2′-F sugar moiety, a 2′-NMA sugar moiety, and/or a β-D-2′-deoxyribosyl sugar moiety.

34. The method of any one of claims 1-33, wherein the internucleoside linkage of Formula I or Formula XIV is adjacent to a nucleoside comprising a cEt sugar moiety, a 2′-MOE sugar moiety, a 2′-OMe sugar moiety, a 2′-F sugar moiety, a 2′-NMA sugar moiety, and/or a β-D-2′-deoxyribosyl sugar moiety.

35. The method of any one of claims 1-34, wherein the internucleoside linkage of Formula I or Formula XIV is adjacent to a nucleoside comprising an adenine, cytosine, 5-methylcytosine, guanine, thymine, or uracil nucleobase.

36. The method of any one of claims 1-35, further comprising attaching a conjugate group to form a conjugated modified oligonucleotide.

37. The method of claim 36, wherein the conjugate group comprises a cell-targeting moiety.

38. The method of claim 37, wherein the cell-targeting moiety has affinity for TfR.

39. The method of claim 38, wherein the cell-targeting moiety has affinity for asialoglycoprotein receptor (ASGPR).

40. A modified oligonucleotide synthesized by the method of any one of claims 20-39, or an oligomeric compound comprising the modified oligonucleotide.

41. A modified oligonucleotide comprising an internucleoside linkage synthesized by the method of any one of claims 1-19, or an oligomeric compound comprising the modified oligonucleotide.

42. A stabilized composition comprising or consisting essentially of methanesulfonyl azide and sulfolane.

43. The composition of claim 42, further comprising a solvent.

44. The composition of claim 43, wherein the solvent is acetonitrile.

45. The composition of any one of claims 42 to 44, further comprising toluene.

46. The composition of any one of claims 42 to 45, wherein the composition comprises 0.1 to 10 equivalents of sulfolane relative to methanesulfonyl azide.

47. The composition of any one of claims 42 to 45, wherein the composition comprises 1 to 10 equivalents of sulfolane relative to methanesulfonyl azide.

48. The composition of any one of claims 42 to 45, wherein the composition comprises 3 to 6 equivalents of sulfolane relative to methanesulfonyl azide.

49. The composition of any one of claims 42 to 45, wherein the composition comprises 4 to 5 equivalents of sulfolane relative to methanesulfonyl azide.

50. The composition, or an evaporated residue thereof, of any one of claims 42 to 49, wherein the composition has a combustion energy less than 500 J·g⁻¹.

51. The composition, or an evaporated residue thereof, of any one of claims 42 to 49, wherein the composition has a combustion energy less than 300 J·g⁻¹.

52. The composition, or an evaporated residue thereof, of any one of claims 42 to 49, wherein the composition is not explosive upon impact.

53. The composition of claim 42 consisting of methanesulfonyl azide, sulfolane, and acetonitrile.

54. A solid-support-bound oligonucleotide intermediate in contact with the composition of any one of claims 42-53.
